(19) United States
(12) Patent Application Publication
(10) Pub. No.: US 2004/0220098 A1
(43) Pub. Date: Nov. 4, 2004

(54) Inhibin compositions and methods of enhancing fertility and growth

(76) Inventors: Daniel G. Satterlee, Prairieville, LA (US); William C. Fioretti, Grapevine, TX (US); Gary G. Cadd, Grapevine, TX (US)

Correspondence Address: JOHN S. PRATT, ESQ, KILPATRICK STOCKTON, LLP, 1100 PEACHTREE STREET, ATLANTA, GA, 30309, US

(21) Appl. No.: 10/755,203

(22) Filed: Jan. 9, 2004

Related U.S. Application Data
(63) Continuation-in-part of application No. 10/262,581, filed on Sep. 30, 2002.
Application No. 10/262,581 is a continuation of application No. 09/436,805, filed on Nov. 9, 1999, now abandoned.
Application No. 09/436,805 is a continuation of application No. 08/984,776, filed on Dec. 4, 1997, now abandoned.
Application No. 08/984,776 is a continuation of application No. 08/481,633, filed on Jun. 7, 1995, now Pat. No. 5,725,858.
Application No. 08/481,633 is a continuation-in-part of application No. 08/395,554, filed on Feb. 28, 1995, now abandoned.
Application No. 08/395,554 is a continuation-in-part of application No. 08/202,964, filed on Feb. 28, 1994, now abandoned.
(60) Provisional application No. 60/439,204, filed on Jan. 10, 2003.

(51) Int. Cl. A61K 38/27; A01K 31/19

(52) U.S. Cl. 514/12; 119/300

(57) ABSTRACT

The present invention relates, in general, to a method of enhancing the fertility and/or growth rate of animals, particularly avians, by administering to a bird a composition comprising a heterologous protein comprising inhibin protein, or a fragment thereof, and a carrier protein, in an acceptable carrier. The present invention also relates to a method of enhancing the fertility and/or growth rate of avians, by administering to a bird a composition comprising a fusion gene product comprising a gene encoded for the expression of alpha-subunit avian inhibin protein, or a fragment thereof, and a gene encoded for the expression of a carrier protein, in an acceptable carrier. An effective amount of the heterologous protein or fusion gene product is administered to an animal such that an immunological response occurs in the animal against the heterologous protein.

### CROSS REFERENCE TO RELATED APPLICATIONS

This is a continuation-in-part application of U.S. patent application Ser. No. 10/262,581, filed Sep. 30, 2002 (incorporated herein by reference), which is a continuation application of U.S. patent application Ser. No. 09/436,805 filed Nov. 9, 1999, which is a continuation application of U.S. patent application Ser. No. 08/984,776 filed Dec. 4, 1997, which is a continuation application of U.S. patent application Ser. No. 08/481,633 filed Jun. 7, 1995 which is a continuation-in-part application of U.S. patent application Ser. No. 08/395,554 filed Feb. 28, 1995 (incorporated herein by reference), which is a continuation-in-part of U.S. patent application Ser. No. 08/202,964, filed Feb. 28, 1994, (incorporated herein by reference). This application further claims priority to U.S. patent application Ser. No. 60/439,204, filed Jan. 10, 2003, which is incorporated herein by reference.

### FIELD OF THE INVENTION

The present invention relates, in general, to a method of enhancing the fertility and/or growth of avians, by administering to a bird a composition comprising a heterologous protein comprising inhibin protein, or a fragment thereof, and a carrier protein in an acceptable vehicle. The present invention also relates to a method of enhancing the fertility and/or growth of avians, by administering to a bird a composition comprising a fusion gene product comprising a gene encoding for the expression of alpha-subunit avian inhibin protein, or a fragment thereof, and a gene encoding for the expression of a carrier protein in an acceptable carrier.

### BACKGROUND OF THE INVENTION

An enormous industry exists which encompasses avians, especially the more established poultry species, primarily egg-type chickens (Single Comb White Leghorns), meat-type chickens (broilers), turkeys, ducks, geese, and quail. Worldwide demand for poultry meat and egg products is great and has been steadily increasing during the last decade. There is a steady trend of rising poultry meat consumption. This trend of increased poultry consumption, such as chicken and turkey meat products is expected to continue in the future parallel to the anticipated growth in human population. The trend of increased poultry consumption is directly related to the fact that poultry meats are considered to be “heart-healthy” foods (low in animal fat content) and poultry competes well with the more expensive red-meats (such as beef, pork, and lamb). Therefore, a method of enhancing growth rates and/or fertility of avians will greatly accelerate the growth of the market and decrease the cost of producing eggs and meat for consumption.

Enhancing growth rates and/or fertility of avians, particularly poultry birds, would be of significant economic value to an industry currently enjoying high growth due to an ever increasing product demand. To satisfy consumer demand and maintain their competitive edge in meat pricing, poultry birds such as broiler and turkey breeders will continue to be in the business of producing as many offspring as possible. Therefore, any method capable of increasing fertility and/or growth rates by even small amounts would generate significant economic benefits, since such a method would decrease the time and cost required to raise a bird to the point of harvest for consumption. Even very small enhancements in fertility and/or growth rates are magnified when one considers the size of the bird populations that can be affected.

Recently, the hormone inhibin has been studied as a potential means for increasing ovulation in mammals. Inhibin is a peptide hormone primarily produced by the gonads, and more particularly by growing follicles and testes. In mammals, it functions as an inhibitory feedback regulator of pituitary follicle-stimulating hormone (“FSH”) secretion. While inhibin's existence was first postulated over 60 years ago, its chemical isolation was only recently achieved.

Mammalian inhibin is a dimeric protein hormone which is composed of an α-subunit (molecular weight 18,000) and a β-subunit (molecular weight 14,000). The α-subunit is unique to inhibin as dimers of the 13-subunit form activin, a hormone which releases FSH from the pituitary gland. The β-subunit exists in two forms (β_{A} and β_{B}), which are distinct but quite similar. Therefore, depending on the β-subunit involved, inhibin exists as inhibin-A or inhibin-B. Both subunits α and β, when joined by disulfide bonds, are required for biological activity in suppressing follicle-stimulating hormone (“FSH”) secretion from the pituitary. The amino acid sequence of the α-subunit of inhibin exhibits approximately 80-90% similarity among the porcine, bovine, human, murine, and domestic chicken species. Excellent reviews on the isolation, production, assay, and biological actions of inhibin are available in Risbridger et al., Current Perspectives of Inhibin Biology, Acta Endocrinologica (Copenh), 122:673-682, (1990); and Rivier, C., et al., Studies of the Inhibin Family of Hormones: A Review, Hormone Research, 28: 104-118 (1987), which are hereby incorporated by reference.

In mammals and birds, FSH plays a role in follicular growth and development, while luteinizing hormone (“LH”) is believed to induce ovulation. Several brain and gonadal factors (peptide and steroid hormones) interact to control gonadotropin hormone release. Of these factors, gonadotropin-releasing hormone (“GnRH”) and inhibin exert opposite controls on pituitary FSH secretion in mammals. Gonadotropin-releasing hormone is a brain decapeptide which acts to stimulate FSH and LH secretion, while inhibin is a gonadal protein which apparently acts to selectively inhibit FSH secretion in mammals.

A basic knowledge of the avian ovulatory process is needed to understand the role of inhibin in the endocrine control of ovulation in birds. Growing follicles on the functionally mature ovary of the domestic hen exist in a distinct size hierarchy. A typical ovary contains four to six large, two to four centimeter in diameter, yolk-filled follicles (F₁ to F₄, F₆), accompanied by a greater number of smaller, two to ten millimeter, yellow follicles, and numerous very small white follicles. The largest preovulatory follicle (F₁) is destined to ovulate the next day, the second largest (F₂) on the following day (approximately 26 hours later), and so on. The control of follicular recruitment and development within this hierarchy is poorly understood. Pituitary gonadotropin involvement has been proven, yet the role of inhibin in the control of avian gonadotropin secretion and control of ovulation remains unclear. Despite conflicting data on how levels of FSH fluctuated during the ovulatory cycle, in all cycling mammals studied, immunoneutralization of endogenous inhibin consistently enhanced ovarian follicular development and ovulation rate, regardless of the antigen used or the mammalian species challenged.

As stated above, inhibin involvement in the regulation of reproductive function in avian species remains unclear. Thus far, published reports have been restricted to the reproductive function of inhibin in domestic fowl. The bulk of this literature supports the theory that inhibin likely exerts parallel physiological roles in fowl to those documented in mammals: in hens, inhibin may serve as a regulator of follicular recruitment and/or development. However, in birds, inhibin's involvement in the control of ovulation rate may or may not be through suppression of pituitary FSH secretion. For example, although low egg producing hens have been found to have higher levels of inhibin in plasma and the granulosa cell layers of preovulatory follicles than high egg producing hens, no difference has been found in plasma FSH levels associated with the rate of egg laying. Wang et al., Increase in Ovarian α-Inhibin Gene Expression and Plasma Immunoreactive Inhibin Level is Correlated with a Decrease in Ovulation Rate in the Domestic Hen, General and Comparative Endocrinology, 91, 52-58, (1993). This reference, therefore, suggests that in hens the ovulation rate-related changes in inhibin α-subunit gene expression and plasma immunoreactive inhibin levels do not directly affect ovulation rate through a modulation of plasma FSH levels. Further, in Johnson, P. A., Inhibin in the Hen, Poultry Science, 72:955-958, (1993), a bovine inhibin RIA system was used to successfully assess immunoreactive inhibin in the plasma of hens, however no significant peak of immunoreactive inhibin was detected throughout the ovulatory cycle in spite of a preovulatory surge of LH. Accordingly, the role of inhibin in folliculogenesis in birds remains unclear.

The α-subunit of chicken inhibin has been successfully cloned and sequenced. Wang and Johnson, Complementary Deoxyribonucleic Acid Cloning and Sequence Analysis of the α-Subunit of Inhibin from Chicken Ovarian Granulosa Cells, Biology of Reproduction, 49, 1-6, (1993), which is incorporated herein by reference in its entirety. Comparison of the avian inhibin sequence to known mammalian inhibin α-subunit sequences showed an 86-89% homology. Northern blot analysis using two isolated probes (cINA₆ and cINA₁₂) revealed that the inhibin α-subunit is expressed in chicken ovarian granulosa cells but not in chicken brain, kidney, liver or spleen tissues.

Further, the improvement of the fertility and/or growth rate of all avians, particularly poultry, is needed to increase the amount of poultry produced for consumption and to improve the efficiency of such production, or feed conversion ratio. Accordingly, there remains a need for a composition and method of improving or enhancing growth rates and/or fertility for all poultry, including chickens, turkeys, ducks, quail, and geese, among others.

The need for a composition and method for enhancing growth rate and/or fertility is not limited to birds. There remains a need for an effective composition and method for enhancing growth rate and/or fertility in many animals. For example, there is a continued need for enhancing growth rate and/or fertility in most animals that are raised agriculturally, such as pigs, cows, and sheep. There is also a continued need of enhancing growth rate and/or fertility in fur bearing animals such as mink, fox, otter, ferret, raccoons, and in rodents such as rats, mice, gerbils, and hamsters used as pets and as laboratory research subjects, and there is an increased need for other animals whose hides are used for decorative purposes.

Also, a composition and method for enhancing growth rate and/or fertility is also needed to increase the population of many animals such as exotic or endangered species to avoid their extinction. There is further a continued need for enhancing growth rate and/or fertility in animals used for racing, entertainment, or showing (competitions) such as horses, dogs, cats, zoo animals, and circus animals. As shown by the increased demands for infertility treatment of humans, there is also a need for enhancing growth rate and/or fertility in humans. Accordingly, there remains a need for a composition and method for enhancing growth rate and/or fertility in many animals.

### SUMMARY OF THE INVENTION

The present invention relates, in general, to a method of enhancing the growth rate and/or fertility of animals, by administering to the animal a composition comprising a heterologous protein comprised of inhibin protein, or a fragment thereof, and a carrier protein, in an acceptable carrier. The present invention also relates to a method of enhancing the fertility and/or growth rate of animals, by administering to the animal a composition comprising a fusion gene product comprising a gene encoding for the expression of alpha-subunit inhibin protein, or a fragment thereof, and a gene encoding for the expression of a carrier protein in an acceptable carrier. An effective amount of the composition comprising the heterologous protein or the fusion gene product is administered to an animal such that an immunological response occurs in the animal against the heterologous protein. It is to be understood that the method of the present invention enhances fertility and/or growth rate of animals that produce inhibin. Preferably, the animal is a bird. More preferably, the bird is a poultry bird. More preferably, the bird is a chicken, turkey, duck, goose or quail. Another preferred bird is a ratite, such as, an emu, an ostrich, a rhea, or a cassowary.

The present invention further relates to the above heterologous protein and fusion gene product, and to methods of producing the same. More particularly, the present invention is directed to a composition and method for making a heterologous protein comprising inhibin, or a fragment thereof, and a carrier protein. The inhibin protein, or fragment thereof, can be avian inhibin, mammalian inhibin, piscine inhibin, or reptilian inhibin. The present invention also includes modified inhibin peptides such that individual amino acids may be conservatively substituted with other natural or non-natural amino acids. The carrier protein, includes, but is not limited to, maltose binding protein, thyroglobulin, keyhole limpet hemocyanin, or bovine serum albumin, among others. The preferred carrier protein is maltose binding protein.

The heterologous protein can be either inhibin, or a fragment thereof, conjugated to the carrier protein or inhibin, or a fragment thereof, fused to the carrier protein. The method of producing the fused heterologous protein comprises inserting cDNA which is encoded for expressing inhibin, or a fragment thereof, into a vector which contains coding information for the production of a carrier protein. After inserting the vector into an expression system, the fused heterologous protein is expressed by the system. Preferably, the heterologous protein is comprised of a poultry inhibin, such as chicken inhibin, although it is to be understood that inhibin from other species may be used, such as mammalian inhibin or ratite inhibin, such as ostrich inhibin, emu inhibin, and rhea inhibin.

The present invention is also directed to a method of enhancing growth rate and/or fertility in animals via the administration of a composition comprising the heterologous protein of the present invention which comprises inhibin protein, or a fragment thereof, and a carrier protein. In one embodiment, the method comprises administering an effective amount of the composition to a female animal. In another embodiment, the method comprises administering an effective amount of the composition to a male animal. Preferably, an immunological response occurs in the animal directed against the heterologous protein. More preferably, the immunological response which occurs in the animal is also directed against the inhibin protein produced by the animal (endogenous inhibin).

The present invention is also directed to a fusion gene product comprising a gene encoded for the expression of alpha-subunit inhibin protein, or a fragment thereof, and a gene encoded for the expression of a carrier protein. The gene encoded for the expression of inhibin protein, or fragment thereof, may be encoded to express avian inhibin, mammalian inhibin, piscine inhibin, or reptilian inhibin. The gene encoded for the expression of a carrier protein may be encoded to express maltose binding protein or bovine serum albumin, among others. The preferred gene encoded to express a carrier protein is encoded to express maltose binding protein.

The present invention also relates to a method of enhancing the growth rate and/or fertility of animals, by administering to the animal a composition comprising a fusion gene product comprising a gene encoded for the expression of alpha-subunit inhibin protein, or a fragment thereof, and a gene encoded for the expression of a carrier protein, and an acceptable carrier. More particularly, the present invention further encompasses gene therapy methods whereby compositions comprising DNA sequences encoding inhibin, or fragments thereof, and a carrier protein, in an acceptable vehicle, are introduced into an animal. The fusion gene product of the present invention may be administered directly to the animal, or it may be administered in a vector, or in a cell containing a vector having the fusion gene product therein.

The method of the present invention enhances growth rate and/or fertility in animals which produce inhibin, such as mammals, reptiles, fish, and birds. More particularly, this method enhances fertility and/or growth rate in poultry, galliformes and ratites. More particularly, this method enhances growth rate and/or fertility in chickens, turkeys, quail, ducks, geese, ostriches, emus, and rhea. This method also enhances growth rate and/or fertility in turtles, including endangered turtle species. Unexpectedly, the method of the present invention increases the onset of puberty or egg lay in animals. In avians, the method of the present invention also improves the feed conversion ratio of a bird and decreases the cost of raising a bird until the time of harvest for meat consumption.

The method of the present invention also improves growth rate and/or fertility in male animals that produce inhibin, such as mammals, reptiles, and birds. More particularly, the method of the present invention increases testosterone levels in male animals. Similarly, the method of the present invention increases the onset of puberty or sperm production in male animals. The method also causes increase in testes growth rate. The method also increases testes weight. The method also accelerates the increase of plasma testosterone levels during growth and puberty of males. Also, the method of the present invention accelerates the onset of maximum sperm production in a male animal. Further, the method of the present invention unexpectedly increases the intensity of sperm production (sperm count) by a male animal. The method of the invention further improves the lifetime fertilization capacity in males. The method of the invention further increases the lifetime sperm production in males. The method of the invention also improves copulation efficiency in males. Further still, the method of the present invention results in delay in reproductive senescence of male birds. Delay in reproductive senescence may include, but is not limited to, any of the following, or combinations thereof: delaying the decline of testes weight in older males, delaying the decline of plasma testosterone levels in older males, delaying the decline of sperm production in older males, and prolonging the persistence of maximum sperm production in animals. Also, the invention includes methods of improving sperm viability in animals. The invention also includes methods of increasing sperm motility, sperm mobility, and combinations thereof. Still further, the method unexpectedly reduces or eliminates the effect of adverse conditions on sperm production of animals exposed to such conditions. Such adverse conditions include elevated temperatures, overcrowding, poor nutrition, and noise. The method of the present invention also surprisingly increases the libido, and therefore, the reproductive potential, of a male bird. The invention also includes methods of increasing somatic growth rate and rate of attaining maximum body weight in avians.

As stated above, the method of the present invention is used to enhance growth rates and/or fertility of any animal that produces inhibin, including, but not limited to, most animals that are raised agriculturally, such as pigs, cows, sheep, turkeys, quail, ducks, geese, chickens, and fish; in fur bearing animals such as mink, fox, otter, ferret, rabbits and raccoon; laboratory animals such as rats, mice, gerbils, and guinea pigs; for animals whose hides are used for decorative purposes such as alligators and snakes; exotic or endangered species; animals used for racing, entertainment, or showing (competitions) such as horses, dogs, cats, zoo animals, and circus animals; and humans. Additional avians that the method of the present invention enhances growth rate and/or fertility thereof include ratites, psittaciformes, falconiformes, piciformes, strigiformes, passeriformes, coraciformes, ralliformes, cuculiformes, columbiformes, galliformes, anseriformes, and herodiones. More particularly, the method of the present invention may be used to enhance growth rates and/or fertility of an ostrich, emu, rhea, chicken, turkey, ducks, geese, quail, partridge kiwi, cassowary, parrot, parakeet, macaw, falcon, eagle, hawk, pigeon, cockatoo, song bird, jay bird, blackbird, finch, warbler, canary, toucan, mynah, or sparrow.

Accordingly, it is an object of the present invention to provide an composition comprising inhibin, or a fragment thereof, and carrier protein, combined with an acceptable carrier, that induces an immunological response in an animal upon administration to the animal.

Accordingly, it is an object of the present invention to provide an composition comprising a heterologous protein comprising inhibin, or a fragment thereof, and carrier protein combined with an acceptable carrier, that induces an immunological response in an animal upon administration to the animal.

It is another object of the present invention to provide an composition comprising a fused heterologous protein comprising inhibin or a fragment thereof and carrier protein combined with an acceptable carrier that induces an immunological response in an animal upon administration to the animal.

Another object of the present invention is to provide an composition comprising a fused heterologous protein comprising inhibin or a fragment thereof and carrier protein combined with an acceptable carrier that induces an immunological response in an animal upon administration to the animal.

Accordingly, it is an object of the present invention to provide an composition comprising a conjugated heterologous protein comprising inhibin or a fragment thereof and carrier protein combined with an acceptable carrier that induces an immunological response in an animal upon administration to the animal.

It is a further object of the present invention to produce an immunological response directed against the heterologous protein of the present invention by direct injection of a composition comprising the fused gene product of the present invention and an acceptable carrier into an animal.

Yet another object of the invention is to provide compositions and methods useful for gene therapy for the modulation of inhibin levels.

It is another object of the present invention to provide a method for enhancing growth rate and/or fertility in animals.

It is an object of the present invention to provide a method for enhancing growth rate and/or fertility in birds.

It is an object of the present invention to provide a method for enhancing growth rate and/or fertility in poultry.

Another object of the present invention is to provide a method for enhancing growth rate and/or fertility in chickens.

It is another object of the present invention to provide a method for enhancing growth rate and/or fertility in turkeys.

Another object of the present invention is to provide a method for enhancing growth rate and/or fertility in quail.

It is also an object of the present invention to provide a method for enhancing growth rate and/or fertility in geese.

It is another object of the present invention to provide a method for enhancing growth rate and/or fertility in ducks.

It is yet another object of the present invention to provide a method for enhancing growth rate and/or fertility in reptiles.

Is another object of the present invention to provide a method for enhancing growth rate and/or fertility in mammals.

Is another object of the present invention to provide a method for enhancing growth rate and/or fertility in fish.

These and other objects, features and advantages of the present invention will become apparent after a review of the following detailed description of the disclosed embodiments and the appended claims.

### BRIEF DESCRIPTION OF THE DRAWINGS

FIG. 1 is an SDS-PAGE gel wherein A is ostrich anti-(chicken inhibin-maltose binding protein) antibodies, B is plasmid pMAL™-c vector standard, C is protein molecular weight standards, D is the actual pMAL™-c vector used in the preparation of the fused heterologous protein, E is the purified fused chicken inhibin-maltose binding protein (heterologous protein) of the present invention, and F is eluent from a purification that was not loaded with the heterologous protein.

FIG. 2 depicts average body weight (mean+standard error of the mean) of chickens receiving 0, 1, 3, or 5 mg of MBP-cINA₅₂₁. Average body weights are shown at 24, 28, and 39 weeks of age. Letters (a, b) above error bars indicate significant differences between groups with different letters at the p level shown.

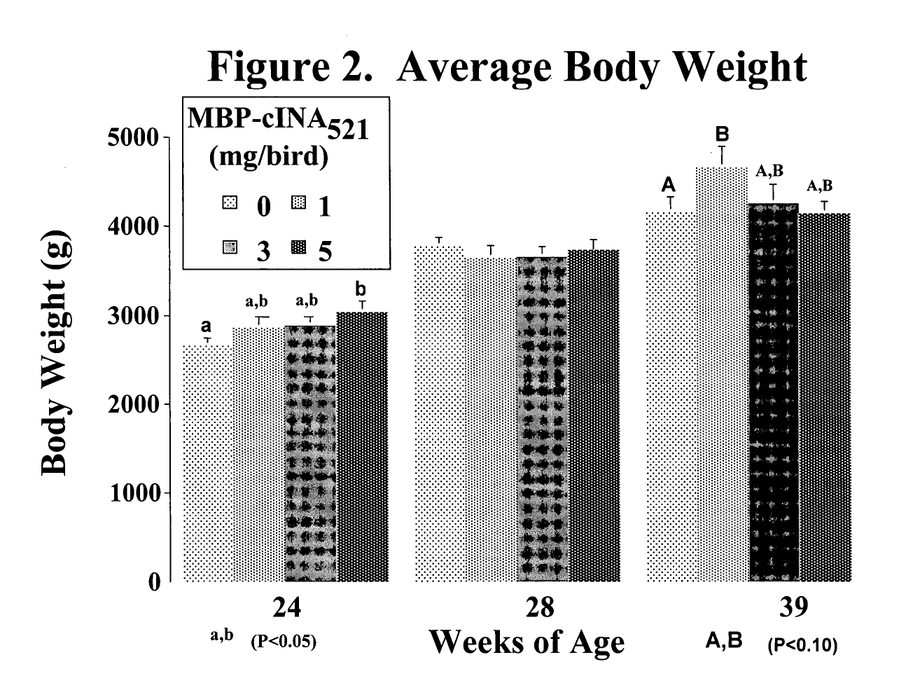

FIG. 3 depicts average total testes weight (mean+standard error of the mean) of chickens receiving 0, 1, 3, or 5 mg of MBP-cINA₅₂₁. Average body weights are shown at 24, 28, and 39 weeks of age. Letters (a, b) above error bars indicate significant differences between groups with different letters at the p level shown.

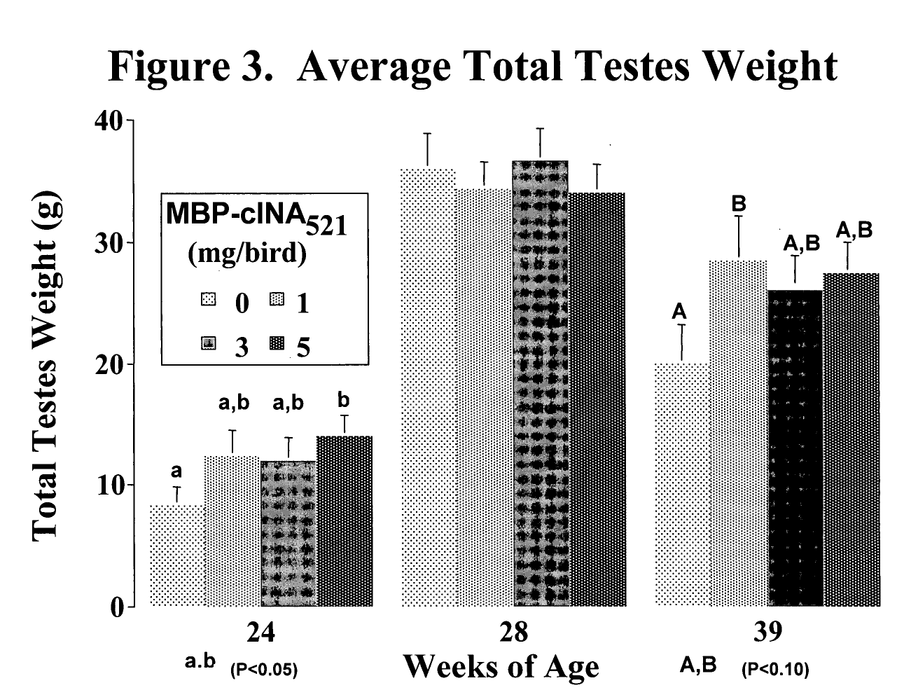

FIG. 4 displays and compares an individual testis at 24 weeks of age from a control animal (4.70 gm) and from an animal receiving 5 mg of MBP-cINA₅₂₁ (5.90 gm).

### DETAILED DESCRIPTION

The present invention relates, in general, to a method of enhancing the growth rate and/or fertility of animals, by administering to the animal a composition comprising a heterologous protein comprising inhibin protein, or a fragment thereof, and a carrier protein, with an acceptable carrier. The present invention also relates to a method of enhancing the growth rate and/or fertility of animals, by administering to the animal a composition comprising a fusion gene product comprising a gene encoded for the expression of alpha-subunit inhibin protein, or a fragment thereof, and a gene encoded for the expression of a carrier protein and an acceptable carrier. A preferred animal of the present invention is an avian animal, more preferably a poultry bird.

An effective amount of the heterologous protein or fusion gene product is administered to an animal such that an immunological response occurs in the animal against the heterologous protein. It is to be understood that the method of the present invention enhances growth rate and/or fertility of animals that produce inhibin. Preferably, the animal is a bird. Preferably, the bird is a poultry bird. More preferably, the bird is a chicken. Another preferred bird is a turkey. Another preferred bird is a quail. Another preferred bird is a goose. Another preferred bird is a duck. Another preferred bird is a goose. Yet another preferred bird is a ratite, such as, an emu, an ostrich, a rhea, or a cassowary. The present invention further relates to the above heterologous protein and fusion gene product, and to a method of producing the same.

After the following definitions, the composition of the present invention is described in detail, followed by a detailed description of the methods of the present invention.

Definitions

The term “bird” or “fowl,” as used herein, is defined as a member of the Aves class of animals which are characterized as warm-blooded, egg-laying vertebrates primarily adapted for flying. Poultry are preferred birds of the present invention, including but not limited to chickens, quail, turkeys, geese, and ducks. The term “chicken” as used herein denotes both chickens used for egg production, such as Single Comb White Leghorns, and chickens raised for consumption, or broilers. The term “Ratite,” as used herein, is defined as a group of flightless, mostly large, running birds comprising several orders and including the emus, ostriches, kiwis, and cassowaries. The term “Psittaciformes”, as used here, include parrots, and are a monofamilial order of birds that exhibit zygodactylism and have a strong hooked bill. A “parrot” is defined as any member of the avian family Psittacidae (the single family of the Psittaciformes), distinguished by the short, stout, strongly hooked beak.

The term “egg” is defined herein as a large female sex cell enclosed in a porous, calcareous or leathery shell, produced by birds and reptiles. “Egg production by a bird or reptile”, as used herein, is the act of a bird laying an egg, or “oviposition”. The term “ovum” is defined as a female gamete, and is also known as an egg. Therefore, egg production in all animals other than birds and reptiles, as used herein, is defined as the production and discharge of an ovum from an ovary, or “ovulation”. Accordingly, it is to be understood that the term “egg” as used herein is defined as a large female sex cell enclosed in a porous, calcareous or leathery shell, when it is produced by a bird or reptile, or it is an ovum when it is produced by all other animals.

The terms “onset of egg lay”, “first egg lay” and “puberty”, in reference to birds are used interchangeably herein, and denote when a bird lays its first egg. Accordingly, “accelerating the onset” of egg lay or puberty in avians, as used herein, denotes inducing an earlier date of first egg lay than a bird would normally have. Similarly, “puberty” and “onset of sperm production” in males are used interchangeably.

The terms “enhancement of fertility,” “enhancing fertility,” and “enhanced fertility” are used to denote an improvement in one or more of the following areas: accelerated onset of puberty (egg lay or ovulation in females; sperm production in males); accelerated onset of maximum egg lay or ovulation in females or accelerated onset of maximum sperm production in males; increase in testes growth rate in males; increase in testes weight in males; acceleration of the increase of plasma testosterone levels during growth and puberty in males; increased intensity of production of eggs in females, or of sperm in males; increased daily sperm production, improved copulation efficiency in males; increased lifetime fertilization capacity in males; increased lifetime sperm production in males; delay in reproductive senescence of birds (which may include, but is not limited to, any of the following, or combinations thereof: delaying the decline of testes weight in older males, delaying in the decline of plasma testosterone levels in older males, delaying the decline of sperm production in older males, and prolonged persistence of egg lay in females or of sperm production in males); increased total lifetime egg lay or ovulation in females; improved feed conversion ratios; improved egg shell quality; decrease in the decline of egg shell quality with age; increased percentage of eggs laid having a medium or larger size; increased percentage of eggs laid having a weight of at least about 48 grams; improved resistance to adverse conditions such as elevated temperatures, overcrowding, poor nutrition, and noise; improved sperm viability in males; improved sperm motility in males; improved sperm mobility in males; increased testosterone production in males; increased ejaculate volume; and increased libido in males.

The phrase “intensity of egg lay” is known to those of ordinary skill in the art to denote frequency of egg lay.

The phrase “lifetime total egg lay” of a bird is defined as the total number of eggs laid by a bird during its entire life span. The phrase “hen day egg production” or “HDEP”, as used herein, is defined as the number of eggs laid by a particular group of hens per day.

The phrase “accelerated onset of maximum egg lay” or “accelerated onset of maximum egg production” as used herein, denotes that the period of time from birth or hatching to when the animal lays eggs or ovulates at 50% of its peak lay rate or ovulation rate, is shorter than the normal period of time from birth to maximum egg lay.

The phrase “increased growth rate” means an enhancement of the somatic growth of an animal. The method of the present invention also increases growth rate in birds. Increased growth rate per unit time may be demonstrated by any number of measures known to one of skill in the art including, but not limited to, somatic growth rate, increase in the amount of meat or muscle mass, alterations in skeletal growth and body weight. In some embodiments, the method increases the rate at which an animal grows prior to and/or during puberty. In some embodiments, the method increases the rate at which the amount of meat or muscle on the animal increases during the period prior to or during puberty. In a preferred embodiment, the animal is a poultry bird, examples of which include, but are not limited to, meat-type chickens, egg-type chickens, turkeys, ducks, and geese. In one embodiment, the method increases the rate at which muscle mass is added during the first 6-8 weeks of the life of a meat-type chicken. In another embodiment, the method increases the rate at which muscle mass is added during the first 15 weeks of the life of a meat-type chicken. In another embodiment, the method increases the rate at which muscle mass is added during the first 25 weeks of the life of a meat-type chicken. The foregoing are non-limiting examples, and the method includes any type of increase in one or more measure of growth during any stage of the life of any animal.

A heterologous protein, as used herein, is defined as a protein comprised of inhibin protein, or a fragment thereof, and a carrier protein. It is to be understood that the terms “inhibin” and “fragment of inhibin” are used interchangeably in the heterologous protein composition, the method of making the heterologous protein, and the method of using the heterologous protein of the present invention.

It is also to be understood that “cINA₅₂₁”, as used herein, denotes a 521 base pair sequence (SEQ ID NO:1). cINA₅₂₁ codes for a portion of the alpha-inhibin subunit of a chicken, represented by SEQ ID NO:2. As used herein, “MBP-cINA₅₂₁” is the heterologous protein that is expressed from a recombinant host cell, after cloning cINA₅₂₁ into a recombinant host cell and expressing a fused heterologous protein comprising maltose binding protein (“MBP”) and the inhibin protein alpha-subunit fragment encoded by cINA₅21. Preferably, MBP-cINA₅₂₁ is produced in host E. coli cells after expression of cloned cINA₅₂₁ using the commercially available vector pMAL™-c. Accordingly, “cINA₅₂₁” denotes a nucleotide sequence, and “MBP-cINA₅₂₁” denotes a fused heterologous protein.

A fused heterologous protein, as used herein, is defined as two different proteins fused together. For example, a protein comprised of inhibin protein, or a fragment thereof, fused to a carrier protein. The fused heterologous protein is expressed from an expression system comprising a fused gene product which contains a gene encoded for the expression of inhibin protein, or a fragment thereof, fused to a gene encoded for expression of a carrier protein. “Fused gene product”, as used herein, is defined as the product resulting from the fusion of the gene encoded for the expression of inhibin protein, or a fragment thereof, and the gene encoded for the expression of a carrier protein.

It is to be understood that spacer peptides may optionally be inserted between the carrier protein and the inhibin protein or fragment thereof in the heterologous proteins of the present invention. It is also to be understood that nucleotides encoding for spacer peptides may optionally be inserted between the nucleotide sequences encoding for carrier protein and the nucleotide sequences encoding for inhibin protein or a fragment thereof in the fusion genes of the present invention.

A conjugated heterologous protein, as used herein, is defined as a protein comprised of inhibin protein, or a fragment thereof, conjugated to a carrier protein. The conjugated heterologous protein is produced by a chemical reaction which links the inhibin protein to the carrier protein with a covalent bond.

An immunological response of an animal to a substance that has been administered to the animal, as used herein, is defined as the cell-mediated and/or humoral response of an animal that is specifically directed against the substance.

The term “selectively interact”, as used herein, is defined as where two objects associate with each other by a covalent bond, a noncovalent bond, a hydrogen bond, electrostatically, a receptor-ligand interaction, a enzyme-substrate interaction, or by other binding or attachment means. The association is selective in that the two objects interact in a specific manner, in a specific position, or only with each other.

Inhibin Compositions

The present invention relates in general to a composition used in a method of enhancing fertility and/or growth rate in animals, including birds. The composition is comprised of a heterologous protein comprising inhibin protein, or a fragment thereof, and a carrier protein, administered in an acceptable carrier. The inhibin can be inhibin from any species of animal that produces inhibin. The inhibin includes, but is not limited to, bird inhibin, mammal inhibin, reptile inhibin, amphibian inhibin, or fish inhibin, among others. More specifically, the mammal inhibin includes, but is not limited to, cow inhibin, human inhibin, horse inhibin, cat inhibin, dog inhibin, rabbit inhibin, sheep inhibin, mink inhibin, fox inhibin, otter inhibin, ferret inhibin, raccoon inhibin, donkey inhibin, rat inhibin, mouse inhibin, hamster inhibin, and pig inhibin. The bird inhibin includes, but is not limited to, ostrich inhibin, emu inhibin, rhea inhibin, cassowary inhibin, kiwi inhibin, turkey inhibin, quail inhibin, chicken inhibin, duck inhibin, goose inhibin, and inhibin from members of the order psittaciformes.

A preferred inhibin is avian, or bird, inhibin. A more preferred inhibin is inhibin from a poultry bird, such as a chicken, duck, quail, goose or turkey. Another preferred inhibin is chicken inhibin. A preferred inhibin is ratite inhibin, such as ostrich, emu, or rhea inhibin. Most preferably, the heterologous protein of the present invention comprises alpha-subunit inhibin protein, or a fragment thereof, and a carrier protein.

The inhibin, or fragment thereof, can be isolated from animal fluids, expressed from genetically engineered cells in an expression system, or synthetically produced from a series of chemical reactions. More particularly, the fragment of inhibin includes, but is not limited to the following compositions: α-subunit inhibin; β-subunit inhibin; recombinant DNA derived fragments of α-subunit inhibin or β-subunit inhibin; synthetic peptide replicas of fragments of α-subunit inhibin or β-subunit inhibin; synthetic peptide replicas of the N-terminal sequence of α-subunit inhibin or β-subunit inhibin; fragments of partially purified inhibin from follicular fluid; fragments of endogenous α-subunit inhibin or β-subunit inhibin; and fragments of exogenous α-subunit inhibin or β-subunit inhibin. As stated above, it is most preferable that the fragment of inhibin is alpha (α)-subunit inhibin, or a fragment thereof. By inhibin, it is understood by one of ordinary skill in the art to encompass inhibin with amino acid substitutions that might render it more immunogenic, or more active at a receptor.

The inhibin in the heterologous protein is either fused to or conjugated with the carrier protein as described below. Where the inhibin is fused to the carrier protein, the heterologous protein is a “fused heterologous protein”. Where the inhibin is conjugated to the carrier protein, the heterologous protein is a “conjugated heterologous protein”. A preferred heterologous protein is a fused heterologous protein.

The identity of the carrier protein in the heterologous protein is not a critical aspect of the present invention. Any carrier protein known in the art can be used in the heterologous protein. The carrier proteins that can be used in the present invention include, but are not limited to the following group: maltose binding protein “MBP”; bovine serum albumin “BSA”; keyhole limpet hemocyanin “KLH”; ovalbumin; flagellin; thyroglobulin; serum albumin of any species; gamma globulin of any species; and polymers of D- and/or L-amino acids. A preferred carrier protein is MBP. Another preferred carrier protein is BSA if the heterologous protein will not be administered to a cow or horse. Yet another preferred carrier protein is ovalbumin if the heterologous protein will not be administered to a bird. A preferred carrier protein is MBP. It is preferred that the carrier protein is immunogenic to the animal that it will be administered to. It is to be understood that use of a carrier protein is optional. For example, if the inhibin, or fragment thereof is sufficiently immunogenic, then it is not necessary to use a carrier protein.

Any adjuvant system known in the art can be used in the composition of the present invention. Such adjuvants include, but are not limited to, Freund's incomplete adjuvant, Freund's complete adjuvant, polydispersed β-(1,4) linked acetylated mannan (“Acemannan”), Titermax® (polyoxyethylene-polyoxypropylene copolymer adjuvants from CytRx Corporation), modified lipid adjuvants from Chiron Corporation, saponin derivative adjuvants from Cambridge Biotech, killed Bordetella pertussis, the lipopolysaccharide (LPS) of gram-negative bacteria, large polymeric anions such as dextran sulfate, and inorganic gels such as alum, aluminum hydroxide, or aluminum phosphate. Emulsions, including but not limited to water in oil emulsions and water in oil in water emulsions may also be used to administer the MAP compositions of the present invention. Another adjuvant system is Freund's incomplete adjuvant. Yet another adjuvant system is Freund's complete adjuvant.

The terms “acceptable carrier” or “acceptable vehicle” are used herein to mean any liquid including but not limited to water or saline, a gel, salve, solvent, diluent, fluid ointment base, liposome, micelle, giant micelle, and the like, which is suitable for use in contact with living animal or human tissue without causing adverse physiological responses, and which does not interact with the other components of the composition in a deleterious manner.

The compositions of the present invention may conveniently be presented in unit dosage form and may be prepared by conventional pharmaceutical techniques. Such techniques include the step of bringing into association the active ingredient and the carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

Preferred unit dosage formulations are those containing a dose or unit, or an appropriate fraction thereof, of the administered ingredient. It should be understood that in addition to the ingredients, particularly mentioned above, the formulations of the present invention may include other agents commonly used by one of ordinary skill in the art.

The compositions of the present invention may be administered through different routes, such as oral, including buccal and sublingual, rectal, parenteral, ocular, aerosol, nasal, intramuscular, subcutaneous, intradermal, and topical. The compositions of the present invention of the present invention may be administered in different forms, including but not limited to solutions, emulsions and suspensions, microspheres, particles, microparticles, nanoparticles, and liposomes.

The present invention also relates to a method of producing the conjugated heterologous protein of the present invention. Methods of producing conjugated proteins are well known in the art. Methods of conjugating proteins to proteins are fully described in Antibodies, A Laboratory Manual, edited by Ed Harlow & David Lane, Cold Spring Harbor Lab (1988), which is incorporated herein by reference. Additional methods of producing conjugated heterologous proteins, including conjugation reagents, such as dialdehydes, carbodiimides, bisdiazotized benzidine and others, carrier proteins, and immunization schedules are described in detail in Chapter 38, pp. 605-618 and Chapter 42, pp. 665-678, in Section VI, “Preparation of Antibodies” in Neuroendocrine Peptide Methodology, edited by P. Michael Conn, Academic Press, New York, 1989, which is incorporated herein by reference.

Although conjugated proteins may be used in the methods of the present invention, fusion proteins are preferred. More particularly, heterologous proteins that are fused yield a homogeneous product, wherein the different segments of the proteins are always fused in the same position, and the same amount of the segments of the proteins are fused. Also, fused heterologous proteins can be produced uniformly, inexpensively, and in large quantities. In contrast, conjugated heterologous proteins are not as uniform as fused proteins. For example, depending on what proteins are being conjugated, the conjugation reaction may yield a mixture of proteins having one or more conjugations, proteins having conjugations in different locations, or proteins that remain unconjugated. Further, some of the conjugations may render the heterologous protein sterically hindered for its intended use (e.g., the immunogenic portion of the protein is sterically hindered). Also, conjugation reaction conditions and reagents may degrade the proteins produced therein. For example, glutaraldehyde is commonly used in conjugation reactions, and it modifies the conformation of proteins. Further, conjugated proteins are more expensive to produce in large quantities than fused proteins.

The present invention is also directed to a fusion gene product comprising a gene encoded for the expression of alpha-subunit inhibin protein, or a fragment thereof, and a gene encoded for the expression of a carrier protein. The gene encoded for the expression of inhibin protein, or fragment thereof, may be encoded to express avian inhibin, mammalian inhibin, fish inhibin, or reptilian inhibin. The gene encoded for the expression of a carrier protein may be encoded to express maltose binding protein or bovine serum albumin, among others. The preferred gene encoded to express a carrier protein is encoded to express maltose binding protein. The fusion gene product and the method of making the fusion gene product are described more fully below.

Briefly described, the method of producing the fused heterologous protein of the present invention is comprised of the steps of inserting a fusion gene product into a coding region of a plasmid, transformation into a host cell with the plasmid, and expressing the fused heterologous protein from the host cell by methods well known in the art. More particularly, the method of producing the fused heterologous protein comprises inserting cDNA that is encoded for expressing inhibin, or a fragment thereof, into a vector that contains coding information for the production of a carrier protein. After inserting the vector into an expression system, the fused heterologous protein is expressed by the system.

Many methods of making fused heterologous proteins are known in the art. Therefore, any method known in the art can be used to produce the fused heterologous protein of the present invention. Many commercially available vector kits and expression systems can be used to prepare the fused heterologous protein of the present invention. An example of such a commercially available vector kit and expression system is pMAL™-c of New England Biolabs, Beverly Mass. Cytoplasmic expression of the fused heterologous protein occurs in the pMAL™-c system. The method of producing the fused heterologous protein of the present invention from a pMAL™-c kit is fully described below in Examples 1 and 2. Other sources of vector kits and expression systems which can be used to produce the fused heterologous protein of the present invention include, but are not limited to: Pharmacia Biotech of Piscataway, N.J.; and Clonetech, of Palo Alto, Calif.

The present invention further relates to a fusion gene product comprising a gene encoded for the expression of inhibin protein, or a fragment thereof, and a gene encoded for the expression of a carrier protein. Spacer genes are optionally included. The inhibin gene can be from any species of animal that produces inhibin. The inhibin gene can be a bird inhibin gene, a mammal inhibin gene, a reptile inhibin gene, an amphibian gene, or a fish gene, among others. More specifically, the mammal inhibin gene includes, but is not limited to, bovine inhibin gene, human inhibin gene, equine inhibin gene, cat inhibin gene, dog inhibin gene, sheep inhibin gene, mink inhibin gene, fox inhibin gene, otter inhibin gene, ferret inhibin gene, raccoon inhibin gene, rat inhibin gene, mouse inhibin gene, hamster inhibin gene, donkey inhibin gene, and pig inhibin gene. The bird inhibin gene includes, but is not limited to, an ostrich inhibin gene, an emu inhibin gene, a rhea gene, a cassowary inhibin gene, a kiwi inhibin gene, a turkey inhibin gene, a quail inhibin gene, a chicken inhibin gene, a goose inhibin gene, a duck inhibin gene, an inhibin gene from any member of the order psittaciformes, an inhibin gene from any falconiformes, an inhibin gene from any piciformes, an inhibin gene from any strigiformes, an inhibin gene from any coraciformes, an inhibin gene from any ralliformes, an inhibin gene from any passeriformes, an inhibin gene from any cuculiformes, an inhibin gene from any columbiformes, an inhibin gene from any galliformes (domestic fowl), an inhibin gene from any anseriformes (geese, ducks, other water fowl), an inhibin gene from any herodiones, and an inhibin gene from any of the following birds: falcon, eagle, hawk, pigeon, parakeet, cockatoo, macaw, parrot, canary, mynah, toucan, and perching bird (such as, song bird, jay, blackbird, finch, warbler, and sparrow).

A preferred inhibin gene is a bird inhibin gene. A preferred inhibin gene is a poultry bird inhibin gene. A preferred inhibin gene is a chicken inhibin gene, a turkey inhibin gene inhibin gene, a duck inhibin gene, a goose inhibin gene or a quail inhibin gene. Another inhibin gene is a ratite inhibin gene. Another inhibin gene is an ostrich inhibin gene. Another inhibin gene is an emu inhibin gene. Yet another inhibin gene is a rhea inhibin gene.

The chicken inhibin α-subunit cDNA clone (cINA6; Wang and Johnson, Complementary Deoxyribonucleic Acid Cloning and Sequence Analysis of the α-Subunit of Inhibin from Chicken Ovarian Granulosa Cells, Biology Of Reproduction, 49: 453-458, 1993), which is hereby incorporated by reference in its entirety, inserted into the EcoR 1 site of Bluescript (Stratagene, La Jolla, Calif.), was obtained as a gift of P. A. Johnson (Cornell University). The cINA6 clone specifically hybridized to ostrich genomic DNA in Southern assays indicating significant DNA homology between these two species (Chouljenko et al., Expression and purification of chicken α-inhibin as a fusion protein with the E. coli maltose binding protein, Poultry Science, 73 (Suppl. 1): 84, 1994). A DNA fragment (“cINA521”) was excised from the cINA6 clone using Pst I digestion. The cINA₅₂₁ DNA fragment encompassed most of the mature chicken inhibin α-subunit. Although cINA₅₂₁ was excised from the cINA6 clone reported in Wang and Johnson, the sequence obtained, namely SEQ ID NO:1, differs from the DNA sequence published in Wang and Johnson.

The ostrich inhibin α-subunit sequence was obtained by polymerase chain reaction (PCR) methods that are well known in the art. More particularly, the primers were constructed based on the sequence reported in Wang, and were used in a PCR reaction with ostrich genomic DNA: 5′-CTCAGCCTGCTGCAGCGCCC-3′ and 5′-GTGTCGACCGCGCGACGCCGAC-3′. More particularly, the above primers correspond to base pairs 778 to 797 and 1347 to 1326, respectively, of the chicken inhibin α-subunit cDNA clone, cINA6, reported in Wang and Johnson. The PCR-product was digested with PstI endonuclease and subcloned into commercially available vector PUC19 (New England Biolabs). The sequence of the ostrich PstI fragment inhibin gene is identical to the corresponding portion of the chicken alpha inhibin.

As stated above, it is to be understood that the carrier protein is not a critical aspect of the present invention. Therefore, a gene encoded to express any carrier protein can be used in the present invention. The carrier protein gene includes, but is not limited to, genes encoded for expressing the following proteins: maltose binding protein “MBP”; bovine serum albumin “BSA”; keyhole limpet hemocyanin “KLH”; ovalbumin; flagellin; thyroglobulin; serum albumin of any species; gamma globulin of any species; and polymers of D- and/or L-amino acids. A preferred carrier protein gene is a gene encoded to express MBP. Another preferred carrier protein gene is a BSA gene if the resultant heterologous protein will not be administered to a cow or horse. Yet another preferred carrier protein gene is an ovalbumin gene if the resultant heterologous protein will not be administered to a bird. The most preferred carrier protein gene is a gene encoded to express MBP or derivatives thereof. The preferred carrier protein genes code for proteins that will increase both the intensity and duration of the host's immune response to the inhibin protein.

The present invention further relates to a method for making a fusion gene product comprising the step of fusing a gene encoded for the expression of inhibin protein, or a fragment thereof, to a gene encoded for the expression of a carrier protein. Spacer genes are optionally employed. Briefly described, the method of making the fusion gene of the present invention comprises the steps of isolating the desired inhibin complementary DNA (cDNA), producing double strand inhibin DNA, obtaining double strand carrier protein DNA, and fusing the double strand inhibin DNA to the double strand carrier protein DNA in a manner such that the fused DNA enables the expression of a fused heterologous protein comprising the inhibin protein, or a fragment thereof, and the carrier protein.

Many methods of isolating genes and making fusion gene products are known in the art. See, for example, Sambrook, Fritsch & Maniatis, Molecular Cloning, A Laboratory Manual, 2nd Ed., Cold Spring Harbor Laboratory Press, 1989, Vols. I, II, III. Therefore, any method known in the art can be used to produce the fusion gene product of the present invention. Many commercially available vector kits can be used to prepare the fusion gene product of the present invention. An example of such a commercially available vector kit is pMAL™-c of New England Biolabs, Beverly, Mass. The method of producing the fusion gene product of the present invention from a pMAL™-c kit is fully described below in Example 1. Other sources of vector kits which can be used to produce the fused gene product of the present invention include, but are not limited to: Pharmacia Biotech of Piscataway, N.J.; and Clonetech, of Palo Alto, Calif.

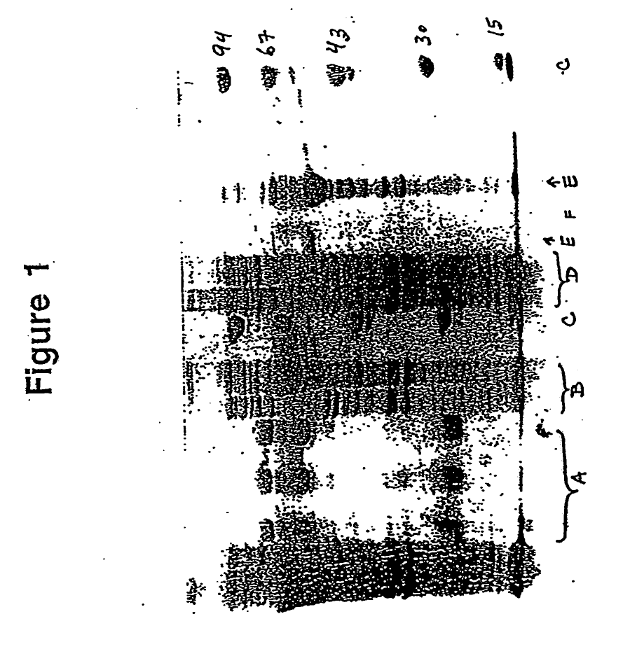

As stated above, the chicken inhibin α-subunit cDNA clone (cINA₆) inserted into the EcoR 1 site of Bluescript was obtained as a gift of P. A. Johnson (Cornell University). A DNA fragment (“cINA₅₂₁”) was excised from the cINA6 clone using Pst I digestion. This fragment (cINA₅₂₁) was cloned in plasmid p-MAL™-c in frame with the maltose binding protein (“MBP”) and a fusion protein of appropriate size (Lane E; FIG. 1) was detected after IPTG (isopropyl β-D-thiogalactopyranoside) induction and SDS-PAGE. The resulting protein conjugate (“MBP-cINA₅₂₁”) was used as an antigen to immunize birds such as pre-pubescent, female Japanese quail (Coturnix coturnix japonica) and chickens against circulating inhibin levels as described in the Examples.

Methods of Enhancing Fertility and/or Growth Rate

It has been unexpectedly discovered that the composition of the present invention enhances the fertility and/or growth rate of animals, and in particular the fertility and/or growth rate of birds. Accordingly, the present invention is also directed to a method of enhancing the fertility and/or growth rate in animals via the administration of compositions comprising the heterologous proteins of the present invention. In one embodiment, the method comprises administering an effective amount of the protein to a female animal such that the fertility and/or growth rate of the animal is increased. In another embodiment, the method comprises administering an effective amount of the protein to a male animal such that the fertility and/or growth rate of the animal is increased. Preferably, an immunological response occurs in the animal directed against the protein. More preferably, the immunological response that occurs in the animal is also directed against the inhibin protein produced by the animal (endogenous inhibin).

More particularly, the method of the present invention comprises the administration of an effective amount of the composition comprising the heterologous protein of the present invention (comprising inhibin, or a fragment thereof, and a carrier protein), and an acceptable carrier, to an animal such that the fertility and/or growth rate of the animal is enhanced. Preferably, the animal is a bird. It is to be understood that a “treated” bird is a bird to which the heterologous protein of the present invention has been administered.

The method of the present invention can be used to enhance the fertility and/or growth rate in any species of female bird that produces inhibin. The female bird includes, but is not limited to, a ratite, a psittaciformes, a falconiformes a piciformes, a strigiformes, a passeriformes, a coraciformes, a ralliformes, a cuculiformes, a columbiformes, a galliformes (domestic fowl), an anseriformes (geese, ducks, other water fowl), and a herodiones. More particularly, the female bird includes, but is not limited to, a turkey, quail, chicken, duck, goose, an ostrich, emu, rhea, kiwi, cassowary, falcon, eagle, hawk, pigeon, parakeet, cockatoo, macaw, parrot, perching bird (such as, song bird, jay, blackbird, finch, warbler, sparrow), and any member of the order psittaciformes. A preferred bird is a poultry bird. Yet another preferred bird is a chicken. Still another preferred bird is a quail. Still another preferred bird is a turkey. Still another preferred bird is a duck. Still another preferred bird is a goose. Another preferred bird is any member of the order psittaciformes. The method of the present invention can also be used to accelerate the fertility and/or growth rate in species of birds that are endangered. Such endangered birds include, but are not limited to, eagles, hawks, condors, and owls.

The inhibin and the carrier protein in the heterologous protein composition of the present invention may vary according to what species of bird the composition will be administered to. It is preferred that avian inhibin and maltose binding protein is used when the composition is to be administered to a bird. A preferred inhibin is domestic chicken or ratite inhibin when the composition is to be administered to a ratite. More preferably, the preferred inhibin is domestic chicken or ostrich inhibin when the composition is to be administered to an ostrich or ratite. Another preferred inhibin is domestic chicken or ostrich inhibin when the composition is to be administered to a chicken. It is to be understood that the inhibin in the heterologous protein need not be from the same species to which the heterologous protein will be administered. For example, a heterologous protein that is administered to an ostrich can be comprised of chicken inhibin and a carrier protein.

It is also to be understood that the composition can further comprise adjuvants, preservatives, diluents, emulsifiers, stabilizers, and other known components that are known and used in vaccines of the prior art. Any adjuvant system known in the art can be used in the composition of the present invention. Such adjuvants include, but are not limited to, Freund's incomplete adjuvant, Freund's complete adjuvant, polydispersed β-(1,4) linked acetylated mannan (“Acemannan”), Titermax® (polyoxyethylene-polyoxypropylene copolymer adjuvants from CytRx Corporation), modified lipid adjuvants from Chiron Corporation, saponin derivative adjuvants from Cambridge Biotech, killed Bordetella pertussis, the lipopolysaccharide (LPS) of gram-negative bacteria, large polymeric anions such as dextran sulfate, and inorganic gels such as alum, aluminum hydroxide, or aluminum phosphate. A preferred adjuvant system is Freund's incomplete adjuvant. Another preferred adjuvant system is Freund's complete adjuvant.

The heterologous protein composition of the present invention can be administered to a bird by any means known in the art. For example, the composition can be administered subcutaneously, intraperitoneally, intradermally, or intramuscularly. Preferably, the composition is injected subcutaneously. The composition can be administered to the bird in one or more doses. Preferably, the composition is administered to the bird in multiple doses wherein an initial immunization is followed by booster immunizations.

The composition can be administered to an animal at any time before the animal ceases to ovulate or produce sperm due to disease or age. The preferred age at which the composition of the present invention is administered to an animal depends upon the species of the animal involved, the mating season (if any) of an animal, and upon the purpose of the administration of the composition.

For example, where the composition is administered to accelerate the onset of egg lay or sperm production, the composition of the present invention is to be administered to a bird before the bird reaches egg lay or puberty. As stated above, the preferred age at which the composition of the present invention is first administered to an animal depends upon the species of the animal involved, the mating season (if any) of an animal, upon the size of the bird, and upon the identity of the components (inhibin and carrier protein) in the composition.

As another example, where the composition is administered to enhance fertility and/or growth rate of agricultural animals that have breeding seasons, the preferred time of administering the composition is prior to the start of the breeding season. In contrast, where the composition is to be administered to a mature animal that has a suppressed egg production rate or a suppressed sperm production rate, then the composition would be administered at the time that the suppression is recognized as problematic.

With respect to an animal having a breeding season, although the heterologous protein of the present invention can be administered to a bird such as a ratite at any age, immunizing the bird during the six months prior to the bird's first breeding season is preferable. It is understood by those of ordinary skill in the art that average female birds initiate egg lay during the first breeding season. It is even more preferable to immunize the bird approximately six months prior to the bird's first breeding season, and then to administer booster immunizations at one month intervals prior to the bird's first breeding season. It is most preferable to immunize the bird approximately six months prior to the bird's first breeding season, and then to administer booster immunizations at one month intervals for six months.

For example, for best results in increasing the fertility and/or growth rate of a female or male ostrich, a primary immunization is administered to the ostrich approximately 6 months before its first breeding season, and then booster immunizations are administered at one month intervals for six months. The primary immunization comprises between approximately 0.5 to 4.5 mg of the heterologous protein of the present invention. The booster immunizations comprise between approximately 0.30 to 3.0 mg of the heterologous protein of the present invention. Preferably, the primary immunization comprises between approximately 1.5 to 3.0 mg of the heterologous protein of the present invention. The booster immunizations comprise between approximately 0.75 to 1.5 mg of the heterologous protein of the present invention. It is also preferable that the heterologous protein is emulsified in Freund's Complete Adjuvant (Sigma Chemical Co., St. Louis, Mo.) in the primary immunization, and that the heterologous protein is emulsified in Freund's Incomplete Adjuvant (Sigma) in the booster immunizations. Even more preferably, the heterologous protein composition is injected subcutaneously. Most preferably, the heterologous protein composition is injected subcutaneously at three sites along the upper thigh region of the ostrich.

The amount administered to a bird of the heterologous protein of the present invention varies according to the species of the bird, the age and weight of the bird, when the protein is administered in relation to the breeding season (if the bird has a breeding season), and how many times the protein is to be administered. Also, the commencement of the administration schedule, or treatment schedule, varies according to the species of the bird, the average age of puberty of that species of the bird, the family history of the bird (with respect to the family's history of age at puberty), the time of year the bird was hatched, the nutritional plane of the bird (highly fed birds come into puberty before those that are undernourished), the general health of the bird at that time of commencement, immunological competence of the bird, the long term health history of the bird, the presence of extreme weather conditions (prolonged excessive inclement weather such as rain, heat, or windiness that the bird is not accustomed to), housing conditions (overcrowding), and a lack of exercise.

One of ordinary skill in the art, in view of the teachings of the present invention, would be able to determine by routine testing the amount of heterologous protein that will be necessary to elicit an immunological response to the protein by the bird.

Another example of the method for enhancing fertility and/or growth rate is as follows. An immunologically effective amount of a conjugated heterologous protein composition is administered to a mammal such that an immunological response occurs in the mammal that is directed against the heterologous protein. The heterologous protein is preferably comprised of mammalian inhibin conjugated to maltose binding protein. Another preferred conjugated heterologous protein is comprised of avian or reptilian inhibin, and maltose binding protein.

For example, the following is a brief summary of the method of the present invention for enhancing fertility and/or growth rate in Japanese Quail as is fully discussed in Example 8. The average age at puberty for an untreated quail is approximately six to eight weeks. The following is a treatment schedule for Japanese quail having an approximate body weight range of 0.1 to 0.25 pounds: primary (first) injection of 0.75 mg of the heterologous protein of the present invention on its 25th day of age; and a booster of 0.375 mg on the 32nd day. Another treatment schedule for Japanese quail having the same body weight is: primary (first) injection of 0.75 mg of the heterologous protein of the present invention on its 25th day of age; and boosters of 0.375 mg on the 32nd, 39th, 46th, 53rd, 60th, and 90th day of age, followed by boosters every 35 days thereafter for three additional challenges (i.e., at 95, 130, and 165 days-of-age.).

More particularly, at 25 days-of-age, 50 female quail were randomly and equally assigned to one of two injection groups (25 birds per group) as follows: (1) MBP-cINA₅₂₁ in Freund's adjuvant (“MBP-cINA₅₂₁/FRN”), or (2) Freund's (adjuvant control; “FRN”). Birds immunized against inhibin (Group 1) were given approximately 0.75 mg MBP-cINA₅₂₁ per bird in the appropriate control vehicle. Equivalent vehicular injection volumes (0.2 mL) of FRN were used in Group 2. All injections were given subcutaneously using tuberculin syringes fitted with 25 gauge needles. As discussed above, booster inhibin immunizations of approximately 0.375 mg MBP-cINA₅₂₁ per bird, or appropriate control challenges, were subsequently administered and the birds were observed for a total of 20 weeks

Beginning at 41 days-of age, which is considered to be day 1 of the egg lay cycle, daily hen-day egg production (“HDEP”) and mortality (“MORT”) measures were recorded for 20 consecutive weeks. In addition, average age at first egg lay (“FIRST”) and age at which hens reached 50% egg production (“FIFTY”) were calculated for each of the treatment groups. As is more fully discussed in Example 8, HDEP, MORT, FIRST, and FIFTY data were subjected to analyses of variance.

Inhibin immunoneutralization clearly accelerated puberty in the quail hens. As shown in Table 1, the average age of FIRST egg lay was decreased (P<0.0088) by nearly six days in inhibin-treated hens. Likewise, as shown in Table 2, the age to FIFTY egg production was markedly reduced (12 days; P<0.01) in inhibin-treated hens.

A positive effect of inhibin treatment on intensity of egg lay was also extant, most notably at the beginning and at the end of the laying cycle. For example, significantly greater (P<0.05) mean HDEP rates were observed in hens treated with MBP-cINA₅₂₁/FRN when compared to the FRN controls during Weeks 1 (16.5 vs 2.6%), 2 (50.0 vs 28.6%), and 4 (96.6 vs 79.7%) and again during Weeks 15 (98.8 vs 86.9%), 16 (96.9 vs 86.3%), 18 (85.7 vs 66.1%), and 20 (96.8% vs 73.8%). Total HDEP rate, inclusive of all 20 weeks of lay, for inhibin-treated hens was 83.5% as compared to 75.4% for the controls (P<0.14).

Besides accelerating puberty, prolonging egg lay, and enhancing the overall intensity of lay, inhibin-treatment decreased the time needed to reach peak egg lay by approximately 3 weeks. MBP-cINA₅₂₁/FRN had HDEP of 96.6% by Week 4 while FRN had HDEP of 96.6% by Week 7. Although differences in peak HDEP values were not statistically evaluated, the treatment differences in mean age at which hens reached 50% HDEP levels (FIFTY) reflect peak performance.

Mortality was not a factor in this study as only eight birds have died (three controls, five treated). The mortality of 16% is within expected limits for quail that have reached 180 days-of age. Most timed biological responses to treatments are studied for effects on onset, magnitude, and duration of response. Herein, the data represent what most would consider to be a full cycle of lay in Japanese quail (i.e., 20 weeks post-initiation of puberty or egg lay). Therefore, the following comments on the effects of inhibin immunoneutralization on the onset, magnitude and duration of egg lay in this species are justified.

The data support the conclusion that onset of puberty was accelerated in the inhibin immunoneutralized group. This was evidenced in the marked treatment differences noted in both the FIRST and FIFTY variables and in the differences observed during the initial weeks of HDEP data.

The acceleration of puberty coupled with the increased persistency of egg lay in the inhibin-challenged birds contributed to an increase in overall HDEP that was marked (8.1%). For example, on a per-hen basis, inhibin treatment essentially translated into a daily gain of approximately 0.081 eggs for every day of the laying cycle that a hen remained viable (i.e., capable of laying an egg). This means that approximately 11 more eggs were obtained for each hen housed during the 20 week period examined (0.081 eggs/hen×140 days-of-lay=11.34 eggs per hen per laying cycle).

Similar results in chickens and turkeys, as found in Coturnix, will have substantial strategic relevance to the poultry industry. It should be noted that Japanese quail have been selected for intensity of egg lay, and that egg laying potential is considered to be even greater in Coturnix than in chicken hens (Single Comb White Leghorns) commercially reared for the single purpose of production of table eggs. Therefore, intensification of egg lay by inhibin vaccination in chickens which have not been selected for egg production but for meat production, e.g., broiler breeders raised for the consumption of their flesh, may be even greater.

Accordingly, the above data shows that the inhibin composition of the present invention enhances fertility as it accelerates the onset of puberty, increases egg lay intensity, and accelerates the onset of maximum egg lay in Japanese Quail. Since Japanese Quail are an acceptable animal model for chickens with respect to their reproductive systems, the above data indicates that the method of the present invention will also accelerate the onset of egg lay in chickens. Accordingly, the method of the present invention will result in an egg producer being able to produce more eggs with lower feed costs.

The above data also shows that the inhibin composition of the present invention enhanced fertility as it minimized the adverse affects of elevated temperatures of the egg lay rate of the Japanese Quail. More particularly, in the eighteenth week of the study described in Example 8, the Quail were inadvertently exposed to elevated temperatures. The birds in Group 1 (treated with MBP-cINAs2₁/FRN) sustained a drop in egg lay rate of approximately 5%. In contrast, the birds in Group 2 (control: FRN) sustained a drop in egg lay rate of approximately 26%. Accordingly, the method of the present invention of enhancing fertility ameliorates the negative impact on egg lay rates of poultry exposed to adverse egg laying conditions. This aspect of the present invention is significant as poultry are often raised in open, uncontrolled environments. Accordingly, poultry stocks are often exposed to adverse conditions such as elevated temperatures, and other extreme weather conditions that they are not acclimated to, which thereby decrease egg lay rates in the poultry industry.

The following is a brief summary of the method of the present invention for enhancing fertility and/or growth rate in ostriches as is discussed in Example 9. The average age at puberty for untreated ostriches is between approximately 28 and 32 months. The following would be the treatment schedule for ostriches having an approximate body weight range of 150 to 300 pounds: primary (first) injection of 5.0 mg of the heterologous protein of the present invention on its 26th month of age; and boosters of 2.5 mg on the 27th, 28th, 30th, 32nd, 34th, and 36th month of age.

The following is a brief summary of the method of the present invention for enhancing fertility and/or growth rate in emu as is discussed in Example 10. The average age at puberty for untreated emu is approximately 20 months. The following would be the treatment schedule for emu having an approximate body weight range of 50 to 90 pounds: primary (first) injection of 3.0 mg of the heterologous protein of the present invention on its 18th month of age; and boosters of 1.5 mg on the 19th, 20th, 22nd, 24th, 26th, and 30th month of age.

The following is a brief summary of the method of the present invention for enhancing fertility and/or growth rate in chickens as is discussed in Example 11. For egg-type chickens, the average age at puberty for an untreated chicken is approximately 20 weeks. The following would be a treatment schedule for an egg-type chicken having an approximate body weight range of 2.0 to 3.5 pounds: primary (first) injection of 1.5 mg of the heterologous protein of the present invention on its 15th week of age; and a booster of 0.75 mg on the 17th week. Another treatment schedule for egg-type chickens having the same body weight is: primary (first) injection of 1.5 mg of the heterologous protein of the present invention on its 15th week of age; and boosters of 0.375 mg on the 17th, 20th, 24th, 30th, 40th, and 50th week of age. For meat-type chickens, the average age at puberty for an untreated chicken is approximately 23-25 weeks. The following would be a treatment schedule for a meat-type chicken having an approximate body weight range of 3.25 to 4.00 pounds at primary injection: primary (first) injection of 1.5 mg of the heterologous protein of the present invention in its 18th week of age; and a booster of 0.75 mg at the 20th week. Another treatment schedule for egg-type chickens having the same body weight is: primary (first) injection of 1.5 mg of the heterologous protein of the present invention on its 18th week of age; and boosters of 0.75 mg on the 20th, 24th, 30th, 40th, and 50th week of age.

The following is a brief summary of the method of the present invention for enhancing fertility and/or growth rate in turkeys as is discussed in Example 12. The average age at puberty for an untreated turkey is approximately 30 weeks. The following would be a treatment schedule for a turkey having an approximate body weight range of 9.0 to 12 pounds: primary (first) injection of 2.0 mg of the heterologous protein of the present invention on its 28th week of age; and a booster of 1.0 mg on the 29th week. Another treatment schedule for turkeys having the same body weight is: primary (first) injection of 2.0 mg of the heterologous protein of the present invention on its 28th week of age; and boosters of 1.0 mg on the 29th, 30th, 34th, 38th, 46th, and 54th week of age.

The following is a brief summary of the method of the present invention for enhancing fertility and/or growth rate in parrots as is discussed in Example 13. The average age at puberty for an untreated parrot is approximately 30 months. The following would be the treatment schedule for a parrot having an approximate body weight range of 0.5 to 1.25 pounds: primary (first) injection of 0.75 mg of the heterologous protein of the present invention on its 28th month of age; and boosters of 0.375 mg on the 29th month. Another treatment schedule for parrots having the same body weight is: primary (first) injection of 0.75 mg of the heterologous protein of the present invention on its 28^{th} month of age; and boosters of 0.375 mg on the 29^{th}, 30th, 32nd, 34th, 36th, and 38th month of age.

The foregoing treatment schedules are simply intended as examples, and all treatment schedules are within the methods of the invention. In some embodiments the first injection occurs as early as the second week. Examples of times for first injections include, but are not limited to, the period beginning week two and ending week four, during the period beginning week two and ending week six, during the period beginning week two and ending week eight, during the period beginning week four and ending week six, during the period beginning week four and ending week eight, and during the period beginning week six and ending week eight, and at approximately week two. While the foregoing schedules may be used with any type of avian, they are one of the preferred embodiments for either egg-type as well as meat-type chickens.

Similarly, any number of booster injections (including but not limited to no booster injection, one booster injection, and more than one booster injections) can be used within method of the present invention. If booster injections are administered, any schedule for administration of booster injections can be used within the present invention. In some embodiments, each booster injection is administered approximately one week after the first injection (or after the previous booster injection, if applicable). In other embodiments, each booster injection is administered approximately two weeks after the first injection (or after the previous booster injection, if applicable). In other embodiments, each booster injection is administered approximately three weeks after the first injection (or after the previous booster injection, if applicable). In other embodiments, each booster injection is administered approximately four weeks to one month after the first injection (or after the previous booster injection, if applicable). In other embodiments, each booster injection is administered approximately six weeks after the first injection (or after the previous booster injection, if applicable). In other embodiments, each booster injection is administered approximately two months after the first injection (or after the previous booster injection, if applicable). In other embodiments, each booster injection is administered approximately three months after the first injection (or after the previous booster injection, if applicable). In other embodiments, each booster injection is administered approximately six months after the first injection (or after the previous booster injection, if applicable).

Similarly, the dose sizes set forth herein are intended as examples, and all dose ranges can be used within the methods of the invention. In some embodiments, the primary dose includes approximately 0.1 mg to approximately 10.0 mg of the heterologous protein. In other embodiments, the primary dose includes approximately 1 to approximately 5 mg of the heterologous protein. In other embodiments, the primary dose includes approximately 0.3 mg to approximately 5.0 mg of the heterologous protein. In other embodiments, the primary dose includes approximately 0.3 mg to approximately 3.0 mg of the heterologous protein. In other embodiments, the primary dose includes approximately 0.3 mg to approximately 2.0 mg of the heterologous protein. In other embodiments, the primary dose includes approximately 0.3 mg to approximately 1.5 mg of the heterologous protein. In other embodiments, the primary dose includes approximately 0.1 mg to approximately 0.5 mg of the heterologous protein. In other embodiments, the primary dose includes approximately 0.5 mg to approximately 1.0 mg of the heterologous protein. In other embodiments, the primary dose includes approximately 0.01 mg to approximately 0.1 mg of the heterologous protein. In other embodiments, the primary dose includes approximately 0.01 mg to approximately 0.05 mg of the heterologous protein. In other embodiments, the primary dose includes approximately 0.05 mg to approximately 0.1 mg of the heterologous protein. In embodiments involving booster injections, any booster dosage can be used. In some embodiments, the booster dosage is the same size as the first dosage. In other embodiments, the booster dosage is approximately 50% to 100% the size of the first dosage. In other embodiments, the booster dosage is approximately 1 to 50% the size of the first dosage. In other embodiments, the booster dosage is approximately 50% the size of the first dosage. In other embodiments, the booster dosage is approximately 25% the size of the first dosage. In other embodiments, the booster dosage is approximately 75% the size of the first dosage. In other embodiments, the booster dosage is approximately 10% the size of the first dosage. Again, the foregoing dose sizes and times are simply examples and any dosage size for first and booster injections can be used.

As discussed above, the method of the present invention enhanced fertility and/or growth rate by accelerating the onset of puberty in the animal that the composition of the present invention was administered to. The term “accelerates” with respect to the onset of egg lay denotes that egg lay of a treated bird commences at least about 3% earlier than egg lay would ordinarily commence in an untreated bird. Preferably, egg lay commences at least about 5% earlier, and more preferably commences at least about 7% earlier. Even more preferably, egg lay commences at least about 10% earlier, and most preferably commences at least about 13% earlier than egg lay would ordinarily commence in an untreated bird.

Also, as discussed above, the method of the present invention enhanced fertility and/or growth rate by increasing egg or sperm production intensity in animals. The term “increases” with respect to egg production denotes that egg production of a treated bird increases at least about 3% with respect to the amount of egg production in an untreated bird. Preferably, egg production increases at least about 7%, and more preferably increases at least about 12%. The term “increases” with respect to sperm production denotes that sperm production of a treated bird increases at least about 10% with respect to the amount of sperm production in an untreated bird. Preferably, sperm production increases at least about 30%, more preferably about 45% and still more preferably increases at least about 60%. In one embodiment, sperm production increases approximately 68%.

Further, as discussed above, the method of the present invention enhances fertility and/or growth rate by accelerating the onset of maximum egg lay or maximum sperm production in an animal. The term “accelerates” with respect to the onset of maximum egg lay denotes that maximum egg lay of a treated bird commences at least about 3% earlier than egg lay would ordinarily commence in an untreated bird. Preferably, maximum egg lay commences at least about 5% earlier, and more preferably commences at least about 7% earlier. Even more preferably, maximum egg lay commences at least about 10% earlier, and most preferably commences at least about 13% earlier than maximum egg lay would ordinarily commence in an untreated bird. The term “accelerates” with respect to the onset of maximum sperm production denotes that maximum sperm production of a treated bird commences at least about 3% earlier than sperm production would ordinarily commence in an untreated bird. Preferably, sperm production commences at least about 5% earlier, and more preferably commences at least about 7% earlier. Even more preferably, sperm production commences at least about 10% earlier, and most preferably commences at least about 13% earlier than sperm production would ordinarily commence in an untreated bird.

Surprisingly, the composition of the present invention is also used to increase the lifetime total egg lay of birds. The term “increase” with respect to total lifetime egg lay denotes that the total lifetime egg lay of a treated bird increases at least about 3% with respect to the total lifetime egg lay of an untreated bird. Preferably, total lifetime egg lay increases at least about 7%, and more preferably increases at least about 12%. Most preferably, total lifetime egg lay increases at least about 15%. The composition of the present invention is also used to increase the lifetime total sperm production of birds. The term “increase” with respect to total lifetime sperm production denotes that the total lifetime sperm production of a treated bird increases at least about 3% with respect to the total lifetime sperm production of an untreated bird. Preferably, total lifetime sperm production increases at least about 7%, and more preferably increases at least about 12%. Most preferably, total lifetime sperm production increases at least about 15%.

Unexpectedly, the composition of the present invention can also be used to decrease or eliminate the need to molt a female bird, e.g., to prolong egg laying persistency by providing for a second cycle of lay. More particularly, if the composition described above is continually administered to the female bird, as disclosed in the method above, the rate of egg lay of the bird, in comparison to if the bird was not treated with the composition of the present invention, would remain high enough so that the bird would not need to be molted to improve its rate of egg lay. It is a common practice in the art to molt a female bird, such as chicken hens (Single Comb White Leghorns, table egg producers), when its egg lay production declines such that the economic cost of maintaining the bird outweighs the economic benefit yielded by the eggs produced. To “molt” a chicken hen, the bird undergoes a period of fasting of approximately four to fourteen days until it beings to molt, e.g., lose its feathers. During the molting period, the bird stops laying eggs. After the bird is placed back onto normal levels of feed, egg production recommences after a period of time. The entire molting period is approximately two months from the beginning of the fast period to the onset of the next egg-lay cycle. In effect, the egg production rate of the bird is rejuvenated. However, after molting a chicken, its rate of egg-lay in the next cycle does not equal the egg production during the first (pre-molt) egg-lay cycle. M. North and D. Bell, Commercial Chicken Production Manual, fourth edition, Chapter 19, Published by Van Norstrand Reinhold of New York.

For example, egg-type chickens reach egg lay at approximately 20 weeks, and produce an economically significant number of eggs for approximately 40 to 50 weeks. At the peak of egg lay, egg-type chickens produce eight to nine eggs every ten days. However, after approximately 50 weeks of egg lay, the rate of egg production decreases to approximately 60% of peak egg lay. At this point, the cost of the feed for the egg-type chicken is greater than the value of the eggs its produces. It is common practice to molt the egg-type chicken at this point, so that when the egg-type chicken recommences egg lay, its rate of egg lay is increased. By “prolonging the persistence of egg lay” with reference to chickens and quail, among other birds, it is meant that egg lay will be prolonged for approximately one to four weeks.

Therefore, the composition of the present invention, as it maintains the rate of egg lay at a higher level than if the bird were not treated with the composition, reduces or eliminates the need to molt a bird. The reduction or elimination of the need to molt a bird results in significant savings. More particularly, during the period that a bird is molted, and prior to that time, the bird has been unproductive with respect to its feed cost before it is molted, and then it is unproductive for a period of time after feeding recommences. Maintaining the rate of egg lay at an enhanced level therefore eliminates or reduces these unproductive phases of the bird, thereby reducing the producer's costs and increasing the producer's profits. Maintaining the rate of egg lay at an enhanced level further enhances egg producer's profits as the rate of egg-lay after molting does not equal the rate of egg-lay in the first cycle of egg lay as discussed above.

Briefly described, the rate of egg lay of birds would be enhanced, thereby avoiding the need to molt the bird, by administering an effective amount of the heterologous protein of the present invention to induce an immunological response thereto, and thereafter administering an effective amount of the heterologous protein (boosters) to maintain a higher than normal rate of egg lay.

Accordingly, the method of the present invention enhances fertility and/or growth rate in male and female animals which produce inhibin, such as mammals, reptiles, and birds such as ratites. More particularly, this method enhances fertility and/or growth rate in poultry birds such as chickens, quail, turkeys, geese and ducks. Unexpectedly, the method of the present invention increases the onset of puberty or first egg lay in animals. Also, the method of the present invention accelerates the onset of maximum egg lay in an animal. Further, the method of the present invention increases the number of eggs laid by an animal. Further still, the method of the present invention prolongs the persistence of maximum egg lay in animals. Still further, the method increases the lifetime total egg lay of an animal.

In avians, the method of the present invention also improves the growth rate of young birds and decreases the time required to feed the bird before harvesting the bird for consumption of meat. In avians, the method of the present invention also improves the growth rate of young birds and decreases the feed conversion ratio of the bird. Also, the method of the present invention unexpectedly reduces or eliminates the effect of adverse laying conditions on egg lay rates of animals exposed to such conditions. Such adverse conditions include elevated temperatures, overcrowding, poor nutrition, and noise.

Although not wanting to be limited by the following, it is theorized that the method of the present invention of enhancing fertility and/or growth rate in animals provides a greater increase in egg production in species that have not been genetically selected for the trait of prolific egg laying. This is particularly true for certain avians. For example, egg-type chickens have been genetically selected for maximum production performance since the late 1920s. (See, for example, Jull, M. A, 1932, Poultry Breeding, John Wiley & Sons.) In terms of the short life span of a chicken, a great deal of selection for this trait occurred over the period of time from approximately 1928 to the present. In contrast, ratites and psittaciformes, most other exotic birds, and to a lesser extent meat-type chickens (broilers) have not been genetically selected for the trait of prolific egg laying. Also, birds that are endangered have also not been genetically selected for the trait of prolific egg laying. Accordingly, as egg-type chickens are already genetically excellent egg layers, the amount of improvement that can be seen with the method of the present invention is limited in comparison to birds that are genetically poor to medium egg layers. Therefore, a much greater amount of improvement in the fertility and/or growth rate is seen with the method of the present invention with birds that have not been genetically selected for prolific egg laying, such as, ratites, psittaciformes, other exotic birds, endangered birds, turkeys, and meat-type chickens.

The immunization of an animal with the heterologous protein of the present invention induces the animal to produce antibodies selectively directed against the heterologous protein. Preferably, the immunization also induces the animal to produce antibodies selectively directed against endogenous inhibin. The production of such antibodies by a bird reduces the time to the onset of puberty or egg lay. The production of such antibodies by the animal also enhances the animal's egg production capability or sperm production capability as the antibodies neutralize the biological activity of inhibin in the animal's blood stream.

Unexpectedly, the method of the present invention also improves fertility and growth rate in male animals that produce inhibin, such as mammals, reptiles, and birds. More particularly, the method of the present invention increases testosterone levels in male animals. Similarly, the method of the present invention increases the onset of puberty or sperm production in male animals. The method also causes increases in testes growth rate. The method also causes increases in testes weight. The method also accelerates the increase of plasma testosterone levels during growth and puberty of males. Also, the method of the present invention accelerates the onset of maximum sperm production in a male animal. Further, the method of the present invention increases the intensity of sperm production (sperm count) by a male animal. Further, the method of the present invention increases the intensity of daily sperm production (sperm count) by a male animal. Further still, the method of the present invention results in delay in reproductive senescence of birds. Delay in reproductive senescence may include, but is not limited to, any of the following, or combinations thereof: delaying the decline of testes weight in older males, delaying in the decline of plasma testosterone levels in older males, delaying the decline of sperm production in older males, and prolonging the persistence of maximum sperm production in animals. Also, the method of the present invention increases ejaculate volume in male animals. Further, the method improves sperm viability in animals. The method also improves sperm mobility in animals. The method further improves sperm motility in animals. The method also improves copulation efficiency in males. Still further, the method unexpectedly reduces or eliminates the effect of adverse conditions on sperm production of animals exposed to such conditions. Such adverse conditions include elevated temperatures, overcrowding, poor nutrition, and noise. The method of the present invention also unexpectedly increases the libido, and therefore, the reproductive potential, of a male bird. The method also increases the lifetime fertilization capacity of a male bird. The method also increases the lifetime sperm production in males

The method of the present invention also increases growth rate in birds. Increased growth rate per unit time may be demonstrated by any number of measures known to one of skill in the art including, but not limited to, somatic growth rate, increase in the amount of meat or muscle mass, alterations in skeletal growth and body weight. In some embodiments, the method increases the rate at which an animal grows prior to and/or during puberty. In some embodiments, the method increases the rate at which the amount of meat or muscle on the animal increases during the period prior to or during puberty. In a preferred embodiment, the animal is a poultry bird, examples of which include, but are not limited to, meat-type chickens, egg-type chickens, turkeys, ducks, and geese. In one embodiment, the method increases the rate at which muscle mass is added during the first 6-8 weeks of the life of a meat-type chicken. In another embodiment, the method increases the rate at which muscle mass is added during the first 15 weeks of the life of a meat-type chicken. In another embodiment, the method increases the rate at which muscle mass is added during the first 25 weeks of the life of a meat-type chicken. The foregoing are non-limiting examples, and the method includes any type of increase in one or more measure of growth during any stage of the life of any animal.

Gene Therapy using the Fusion Gene Product

The present invention also relates to a method of enhancing the growth rate and/or fertility of animals, by administering to the animal a composition comprising a fusion gene product comprising a gene encoded for the expression of alpha-subunit inhibin protein, or a fragment thereof, and a gene encoded for the expression of a carrier protein, in an acceptable carrier. The fusion gene product of the present invention may be administered directly to the animal, or it may be administered in a vector, or in a cell containing a vector having the fusion gene product therein.

Various methods of transferring or delivering DNA to cells for expression of the gene product protein, otherwise referred to as gene therapy, are disclosed in Gene Transfer into Mammalian Somatic Cells in vivo, N. Yang, Crit. Rev. Biotechn. 12(4): 335-356 (1992), which is hereby incorporated by reference. Gene therapy encompasses incorporation of DNA sequences into somatic cells or germ line cells for use in either ex vivo or in vivo therapy. Gene therapy functions to replace genes and augment normal or abnormal gene function.

Strategies for gene therapy include therapeutic strategies such as identifying a defective gene and then adding a functional gene to either replace the function of the defective gene or to augment a slightly functional gene; or prophylactic strategies, such as adding a gene for the product protein. As an example of a prophylactic strategy, a fused gene product which encodes for inhibin, or a fragment thereof, and a carrier protein may be placed in an animal thereby secondarily reducing the levels of inhibin in the animal due to the immune response.

Any protocol for transfer of the fused gene product of the present invention is contemplated as part of the present invention. Transfection of promoter sequences, other than one normally found specifically associated with inhibin, or other sequences which would decrease production of inhibin protein are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994.

Gene transfer methods for gene therapy fall into three broad categories-physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the animal. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer. In ex vivo gene transfer, cells are taken from the animal and grown in cell culture. The DNA is transfected into the cells, the transfected cells are expanded in number and then reimplanted in the animal. In in vitro gene transfer, the transformed cells are cells growing in culture, such as tissue culture cells, and not particular cells from a particular animal. These “laboratory cells” are transfected, the transfected cells are selected and expanded for either implantation into an animal or for other uses.

In vivo gene transfer involves introducing the DNA into the cells of the animal when the cells are within the animal. Methods include using virally mediated gene transfer using a noninfectious virus to deliver the gene in the animal or injecting naked DNA into a site in the animal and the DNA is taken up by a percentage of cells in which the gene product protein is expressed. Additionally, the other methods described herein, such as use of a “gene gun,” may be used for in vitro insertion of inhibin DNA or inhibin regulatory sequences.

Chemical methods of gene therapy may involve a lipid based compound, not necessarily a liposome, to ferry the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the animal to provide the gene product from the inserted DNA.

Viral vectors have also been used to insert genes into cells using in vivo protocols. To direct tissue-specific expression of foreign genes, cis-acting regulatory elements or promoters that are known to be tissue specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors that have been used for gene therapy protocols include but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed by the 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most cell types, precise single copy vector integration into target cell chromosomal DNA, and ease of manipulation of the retroviral genome.

The adenovirus is composed of linear, double stranded DNA complexed with core proteins and surrounded with capsid proteins. Advances in molecular virology have led to the ability to exploit the biology of these organisms in order to create vectors capable of transducing novel genetic sequences into target cells in vivo. Adenoviral-based vectors will express gene product peptides at high levels. Adenoviral vectors have high efficiencies of infectivity, even with low titers of virus. Additionally, the virus is fully infective as a cell free virion so injection of producer cell lines are not necessary. Another potential advantage to adenoviral vectors is the ability to achieve long term expression of heterologous genes in vivo.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of the cells which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs.

Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of inhibin may be accomplished by administering compounds that bind to the inhibin gene, or control regions associated with the inhibin gene, or its corresponding RNA transcript to modify the rate of transcription or translation. Additionally, cells transfected with a DNA sequence encoding inhibin, or a fragment thereof, and a carrier protein may be administered to an animal to provide an in vivo source of the heterologous protein of the present invention. For example, cells may be transfected with a vector containing the fusion gene product of the present invention, encoding inhibin, or a fragment thereof, and a carrier protein.

The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. It may be desirable that a recombinant DNA molecule comprising the fused gene product of the present invention is operatively linked to an expression control sequence to form an expression vector capable of expressing the heterologous protein of the present invention. The transfected cells may be cells derived from the animal's normal tissue, the animal's diseased tissue, or may be non-animal cells.

For example, cells removed from an animal can be transfected with a vector capable of expressing the heterologous protein of the present invention, and re-introduced into the animal. The transfected cells then produce the heterologous protein of the present invention thus inducing an immunological response to the inhibin. Cells may also be transfected by non-vector, or physical or chemical methods known in the art such as electroporation, ionoporation, or via a “gene gun.” Additionally, the fused gene product of the present invention may be directly injected, without the aid of a carrier, into an animal. In particular, the fused gene product of the present invention may be injected into skin, muscle or blood.

The gene therapy protocol for transfecting inhibin, or a fragment thereof, into an animal may either be through integration of the fused gene product into the genome of the cells, into minichromosomes, or as a separate replicating or non-replicating DNA construct in the cytoplasm or nucleoplasm of the cell. Heterologous protein expression may continue for a long-period of time, or the fused gene product of the present invention may be reinjected periodically to maintain a desired level of the heterologous protein in the cell, the tissue, or organ, or a determined blood level.

The fused gene product of the present invention can be administered to a bird by any means known in the art. For example, the composition can be administered subcutaneously, intraperitoneally, intradermally, intravascularly, or intramuscularly. Preferably, the composition is injected subcutaneously. Another preferable administration is intravascular infusion near the preferred site of therapy. The composition can be administered to the bird in one or more doses. Preferably, the composition is administered to the bird in multiple doses wherein an initial immunization is followed by booster immunizations. The preferred amount of the fused gene product to be administered is between 50 and 300 micrograms per kilogram of body weight. Preferably, the fused gene product is administered in a carrier, such as a buffer or Freund's adjuvant.

The methods of the present invention for enhancing fertility and/or growth rate in birds will greatly accelerate the growth of the population and therefore the market for poultry.

The utility of the method of the present invention for enhancing fertility and/or growth rate is not limited to enhancing fertility and/or growth rate in birds. The present method for enhancing fertility and/or growth rate can be used in many animals. As stated above, the method of the present invention is used to enhance fertility and/or growth rate of any animal that produces inhibin, including, but not limited to, most animals that are raised agriculturally, such as pigs, cows, sheep, turkeys, quail, ducks, geese, turtles, fish, and chickens; in fur bearing animals such as mink, fox, otter, ferret, rabbits, and raccoons; rodents for laboratory testing such as mice, rats, hamsters, guinea pigs and gerbils; for animals whose hides are used for decorative purposes such as alligators and snakes; exotic or endangered species; animals used for racing, entertainment, or showing (competitions) such as horses, dogs, cats, zoo animals, and circus animals; and humans. Additional avians that the method of the present invention enhances fertility and/or growth rate thereof include ratites, psittaciformes, falconiformes, piciformes, strigiformes, passeriformes, coraciformes, ralliformes, cuculiformes, columbiformes, galliformes, anseriformes, and herodiones. More particularly, the method of the present invention may be used to enhance fertility and/or growth rate of an ostrich, emu, rhea, kiwi, cassowary, parrot, parakeet, macaw, falcon, eagle, hawk, pigeon, cockatoo, song bird, jay bird, blackbird, finch, warbler, canary, toucan, mynah, or sparrow.

One of ordinary skill in the art will understand that the immunoassay techniques that can be used in the above method are well known in the art. Therefore, any immunoassay technique, label, and visualization method known in the art can be used in the above method, including ELISA and radioimmunoassay (RIA). A preferred immunoassay is ELISA (“enzyme linked immunosorbent assay”), and a preferred label is horseradish peroxidase. Another preferred label is a colored latex bead. The colored latex bead can be any color desired for visualization purposes. Preferably, the latex bead is yellow, red, blue, or green. The colored latex bead can be hollow or solid, but it preferably is hollow to minimize its weight. The size of the latex bead varies according to its intended use in immunoassays. One of ordinary skill in the art would be able to ascertain by routine testing the largest bead size that is visible yet does not interfere sterically with the immunoassay reactions. Preferably, the latex bead is less than 0.5μ in diameter, and most preferably it is less than 0.2μ in diameter.

For example, circulating inhibin concentrations in the blood of a bird can be determined using standard sandwich ELISA techniques. First, bind anti-inhibin antibodies that are directed against a portion of inhibin, or a fragment thereof, to the wells of a microtiter plate. After washing and blocking the plate, then add a quantity of blood plasma that was obtained from the bird to be tested. After allowing any inhibin in the sample, if present, to selectively interact with the immobilized anti-inhibin antibody, the sample is washed from the well of the plate. Next, add labeled anti-inhibin antibodies to the well that are directed against a different portion of the inhibin, or a fragment thereof, than the antibody immobilized in the well. The antibody can be labeled with any label known in the art, such as horseradish peroxidase. After allowing the labeled anti-inhibin antibody to selectively interact with any immobilized inhibin, any uninteracted labeled anti-inhibin antibodies are removed by washing. The amount of inhibin present in the plasma sample is determined by using the appropriate visualization means for the label used in the ELISA to quantify the amount of immobilized labeled anti-inhibin antibody in the well. Standard positive and negative controls are to be run simultaneously in neighboring plate wells.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Producing a Fused Gene Product comprising a Gene Encoded for Expressing Chicken Inhibin, and a Gene Encoded for Expressing Maltose Binding Protein.

The following is a method for producing a fused gene product comprising a gene (cINA₅₂₁) encoded for expressing a fragment of alpha-subunit chicken inhibin (SEQ ID NO:2), and a gene encoded for expressing maltose binding protein. The fused gene product of the present invention is made from the pMAL™-c vector kit, from New England Biolabs, Beverly, Mass.

The pMAL™ vectors provide a method for producing a protein expressed from a gene cloned in a reading frame. The cloned gene is inserted downstream from a gene which encodes maltose-binding protein (“MBP”), and results in the expression of an MBP fusion protein (“MBP-cINA₅₂₁”). The method yields high-level expression of the cloned sequences, and a one-step purification for the fusion protein, MBP-cINA₅21, using MBP's affinity for maltose.

The following is a Method of Ligating the Inhibin Gene, cINA₅₂₁, into a pMAL™-c Vector:

1. Digest 0.5 μg pMAL™-c plasmid DNA in 20 μl with the restriction endonuclease PstI.

2. Digest 5 μg of cINA₆ plasmid DNA which contains the chicken inhibin gene with the same enzyme, PstI.

3. Check for complete digestion by running 4 μl of the pMAL™ reaction and 4 μl of the cINA₆ reaction on a 0.8% agarose gel. Then run preparative agarose gel and purify the PstI cINA₅₂₁ fragment by prep-A-Gene purification kit.

4. Add 0.05 Units of calf intestinal alkaline phosphatase (NEB #290) to the vector DNA digestion. Incubate at 37° C. for 1 hour.

5. Add an equal volume of a 1:1 phenol/chloroform mixture to the vector restriction digest, mix, and after centrifuging, remove the aqueous (top) phase and place in a fresh tube. Repeat with chloroform alone.

6. Add 10 μg glycogen or tRNA to the vector digest as carrier, add 1/10th volume 3 M sodium acetate, mix, then add two volumes ethanol. Incubate at −20 C for 30 minutes.

7. Microcentrifuge for 15 minutes. Pour off the supernatant, rinse the pellet with 70% ethanol, and allow to dry.

8. Resuspend each sample in 20 μl of water.

9. Mix: 0.2 μg vector digest; 0.5 μg insert digest; add water, up to 18 μl, then add 2 μl of 10× ligase buffer; 0.5 μl NEB T4 ligase (#202; ˜200 units); and incubate at 16° C. for 2 hours to overnight.

10. Heat at 65° C. for 5 minutes; cool on ice.

11. Mix 5 μl of ligation mixture with 100 μl competent DH5α (or any lacZα-complementing strain) and incubate on ice for 15-30 minutes. Heat to 42° C. for 2 minutes.

12. Add 1 ml LB and incubate at 37° C. for 60 minutes. Spread on an LB plate containing 100 μg/ml ampicillin. Incubate overnight at 37° C. Pick colonies with a sterile toothpick onto a master LB amp plate and an LB amp plate containing 80 μg/ml Xgal and 0.1 mM IPTG. Incubate at 37° C. for 8 to 16 hours. Score the Lac phenotype on the Xgal plate and recover the “white” clones from the master.

13. Screen for the presence of inserts in one or both of the following ways:

A. Prepare miniprep DNA. Digest with an appropriate restriction endonuclease to determine the presence and orientation of the insert.

B. i)Grow a 5 ml culture in LB amp broth to about 2×10⁸/ml.

ii) Take a 1 ml sample. Microcentrifuge for 2 minutes, discard the supernatant and resuspend the cells in 50 μl protein gel SDS-PAGE sample buffer.

iii) Add IPTG to the remaining culture to 0.3 mM, for example 15 μl of a 0.1 M stock solution. Incubate at 37° C. with good aeration for 2 hours.

iv) Take a 0.5 ml sample. Microcentrifuge for 2 minutes, discard the supernatant and resuspend the cells in 100 μl SDS-PAGE sample buffer.

v) Boil the samples 5 minutes. Electrophorese 15 μl of each sample on a 10% SDS-PAGE gel along with a set of protein MW standards and 15 μl of the supplied MBP in SDS-PAGE sample buffer. Stain the gel with Coomassie brilliant blue. An induced band is easily visible at a position corresponding to the molecular weight of the fusion protein. The molecular weight of the MBP alone is 42,000 Daltons.

### EXAMPLE 2

Producing a Fused Heterologous Protein, “MBP-cINA₅₂₁”, Comprising Chicken Inhibin and Maltose Binding Protein.

The following is a method of producing a fused heterologous protein comprising chicken inhibin and maltose binding protein, “MBP-cINA₅₂₁”. The fused gene product of Example 1 expresses the fused heterologous maltose binding protein-inhibin protein, “MBP-cINAs21”, as follows:

1. Inoculate 80 ml rich broth+glucose and ampicillin (see Media and Solutions below) with 0.8 ml of an overnight culture of cells containing the fusion plasmid of Example 1.

2. Grow at 37° C. with good aeration to 2×10⁸ cells/ml (A₆₀₀ of ˜0.5). Take a sample of 1 ml and microcentrifuge for 2 minutes (uninduced cells). Discard supernatant and resuspend the cells in 50 μl SDS-PAGE sample buffer. Vortex and place on ice.

3. Add IPTG (isopropylthiogalactoside) to the remaining culture to give a final concentration of 0.3 mM, e.g. 0.24 ml of a 0.1 M stock in H₂O (see Media and Solutions). Continue incubation at 37° C. for 2 hours. Take a 0.5 ml sample and microcentrifuge for two minutes (induced cells). Discard supernatant and resuspend the cells in 100 μl SDS-PAGE: sample buffer. Vortex to resuspend cells and place on ice.

4. Divide the culture into two aliquots. Harvest the cells by centrifugation at 4000×g for 10 min. Discard the supernatant and resuspend one pellet (sample A) in 5 ml of lysis buffer (see Media and Solutions). Resuspend the other pellet (sample B) in 10 ml 30 mM Tris-Cl, 20% sucrose, pH 8.0 (8 ml for each 0.1 g cells wet weight).

5. Freeze samples in a dry ice-ethanol bath (or overnight at −28° C.). Thaw in cold water (20° C. is more effective than 70° C., but takes longer).

6. Sonicate, monitor cell breakage, by measuring the release of protein using the Bradford assay or the release of nucleic acid at A₂₆₀° until it reaches a maximum. Add 0.6 ml 5M NaCl.

7. Centrifuge at 9,000×g for 20 minutes. Decant the supernatant (crude extract 1) and save on ice. Resuspend the pellet in 5 ml lysis buffer. This is a suspension of the insoluble matter (crude extract 2).

Column Purification of Heterologous Fused Maltose Binding Protein-Inhibin Protein, “MBP-cINA₅₂₁”, as Produced Above, is as Follows:

1. Swell amylose resin (1.5 g) for 30 min. in 50 ml column buffer (see Media and Solutions) in a 250 ml filter flask. De-gas with an aspirator. Pour in a 2.5×10 cm column. Wash the column with 3 column volumes of the same buffer+0.25% Tween 20.

The amount of resin needed depends on the amount of fusion protein produced. The resin binds about 3 mg/ml bed volume, so a column of about 15 ml should be sufficient for a yield of up to 45 mg fusion protein/liter culture. A 50 ml syringe plugged with silanized glass wool can be substituted for the 2.5 cm column. The column height to diameter ratio should be less than or equal to 4.

2. Dilute the crude extract 1:5 with column buffer+0.25% Tween 20. Load the diluted crude extract at a flow rate of [10×(diameter of column in cm)²]ml/hr. This is about 1 ml/min. for a 2.5 cm column.

The dilution of the crude extract is aimed at reducing the protein concentration to about 2.5 mg/ml. If the crude extract is less concentrated, do not dilute it as much. A good rule of thumb is that 1 g wet weight of cells gives about 120 mg protein.

3. Wash with 2 column volumes column buffer+0.25% Tween 20.

4. Wash with 3 column volumes column buffer without Tween 20.

5. Elute the fusion protein, “MBP-cINA₅₂₁”, with column buffer+10 mM maltose+0.1% SDS (optional 10 mM β-mercaptoethanol, 1 mM EGTA). Collect 10-20 3 ml fractions. Assay the fractions for protein, e.g., by the Bradford assay or A₂₆₀°; the fractions containing the fusion protein have easily detectable protein. The fusion protein elutes soon after the void volume of the column.

Media and Solutions

Rich medium+glucose and ampicillin=per liter: 10 g tryplone, 5 g yeast extract, 5 g NaCl, 2 g glucose. Autoclave; add sterile ampicillin to 100 μg/ml.

0.1 M IPTG Stock=1.41 g IPTG (isopropyl-β-o-thiogalactoside); add H₂O to 50 ml. Filter, and sterilize.

0.5 M sodium phosphate buffer, pH 7.2 (stock)=(A) 69.0 g NaH₂PO₄H₂₀ to 1 liter with H₂O.

(B) 70.9 g Na₂HPO₄ to 1 liter with H₂O.

Mix 117 ml (A) with 383 ml (B). The pH of this stock should be 7.2. Diluted to 10 mM in column buffer, the pH should be 7.0.

Lysis Buffer

| Per Liter | Final Concentration |
|---|---|
| 20 ml 0.5 M Na₂HPO₄ | 10 mM phosphate |
| 1.75 g NaCl | 30 mM NaCl |
| 10 ml 25% Tween 20 | 0.25% Tween j20 |
| 0.7 ml β-mercaptoethanol | 10 mM β-ME |
| (“β-ME”) (optional) | |
| 20 ml 0.5 M EDTA (pH 8) | 10 mM EDTA |
| 10 ml 1 M EGTA (pH7) | 10 mM EGTA |
| Adjust to pH 7.0 with HCL or | |
| NaOH | |

Column Buffer

| Per Liter | Final Concentration |
|---|---|
| 20 ml 0.5 M sodium phosphate, | 10 mM phosphate |
| pH 7.2 | |
| 29.2 g NaCl | 0.5 M NaCl |
| 1 ml 1 M sodium azide | 1 mM azide |
| 0.7 mM β-ME (optional) | 10 mM β-ME |
| 1 ml 1 M EGTA (pH 7) (optional) | 1 mM EGTA |
| Adjust to pH 7.0 if necessary. | |

Low Salt Column Buffer

| Per Liter | Final Concentration |
|---|---|
| 20 ml 0.5 M sodium phosphate, | 10 mM phosphate |
| pH 7.2 | |
| 1.75 g NaCl | 30 mM NaCl |
| 1 ml 1 M sodium azide | 1 mM azide |
| 0.7 ml β-mercaptoethanol | 10 mM β-ME |
| (optional) | |
| 1 ml 1 M EGTA (pH 7) (optional) | 1 mM EGTA |
| Adjust to pH 7.0 if necessary. | |

The purity of the fused chicken inhibin-MBP heterologous protein, “MBP-cINA₅₂₁”, after passing through the column is illustrated in FIG. 1, columns “E”. The column marked “F” is the eluent from the column when no heterologous protein has been loaded on the column (the negative control). The columns marked “B” represent the plasmid pMAL™-c vector standards. The columns marked “C” are molecular weight standards. The columns marked “D” are the actual pMAL™-c vector used in the preparation of the fused chicken-inhibin-MBP heterologous protein, “MBP-cINA₅₂₁”, prior to the insertion of the inhibin gene as described in Example 2. The above proteins were electrophoresed on a SDS-PAGE gel in SDS-PAGE sample buffer, and stained with Coomassie brilliant blue stain.

### EXAMPLE 3

Enhancing Fertility in Quail.

As stated above, the chicken inhibin a-subunit cDNA clone (cINA6) inserted into the EcoR 1 site of Bluescript was obtained as a gift of P. A. Johnson (Cornell University). A DNA fragment (“cINA₅₂₁”) was excised from the cINA6 clone using Pst I digestion. The cINA₅₂₁ DNA fragment encompassed most of the mature chicken inhibin α-subunit. This fragment (cINA₅₂₁) was cloned in plasmid p-MAL™-c in frame with the maltose binding protein (“MBP”) and a fusion protein of appropriate size (Lane E; FIG. 1) was detected after IPTG (isopropyl β-D-thiogalactopyranoside) induction and SDS-PAGE. The resulting protein conjugate (“MBP-cINA₅₂₁”) was used as an antigen to immunize pre-pubescent, female Japanese quail (Coturnix coturnix japonica) against circulating inhibin levels as described below.

Hatchling quail were brooded in a Model 2S-D Petersime brooder battery modified for quail. Initial brooding temperature was approximately 37.8 C with a weekly decline of approximately 2.8 C until ambient temperature was achieved. During the growing period (i.e., until approximately 6 wks-of-age), a quail starter ration (28% CP, 2,800 kcal ME/kg of feed) and water were provided for ad libitum consumption, and continuous dim light (22 l×) with a 14 h light (280 to 300 l×):10 hr dark override was used. At 25 days-of-age, 50 quail were randomly and equally assigned to one of two injection groups (25 birds per group) as follows: (1) MBP-cINA₅₂₁ in Freund's adjuvant (“MBP-cINA₅₂₁/FRN”), or (2) Freund's (adjuvant control; “FRN”). Birds immunized against inhibin (Group 1) were given approximately 0.75 mg MBP-cINA₅₂₁ per bird in the appropriate control vehicle. Equivalent vehicular injection volumes (0.2 mL) of FRN were administered to Group 2. All injections were given subcutaneously using tuberculin syringes fitted with 25 ga needles. Following the initial injections, quail were wingbanded to identify them by treatment before housing (individually) in laying cages. Weekly booster inhibin immunizations of approximately 0.375 mg MBP-cINA₅₂₁ per bird, or appropriate control challenges, were subsequently administered for five consecutive weeks (i.e., at 32, 39, 46, 53, and 60 days-of-age) and then every 35 days thereafter for three additional challenges (i.e., at 95, 130, and 165 days-of-age). Beginning at 6 weeks-of-age, a quail breeder ration (21% CP, 2,750 kcal ME/kg of feed) and water were provided for ad libitum consumption.

Beginning at 41 days-of age (considered Day 1 of the egg lay cycle), daily hen-day egg production (“HDEP”) and mortality (“MORT”) measures were recorded for 20 consecutive weeks. In addition, average age at first egg lay (“FIRST”) and age at which hens reached 50% egg production (“FIFTY”), or maximum egg lay as defined above, were calculated for each of the treatment groups.

Hen-day egg production data were subjected to an analyses of variance (“ANOVA”) that incorporated a completely randomized design with a split-plot arrangement of treatments. The main plot consisted of the two injection treatments (MBP-cINA₅₂₁/FRN, or FRN) and the 20 laying periods of 7 days each comprised the split.

Inhibin immunoneutralization clearly accelerated puberty in the quail hens. The average age of FIRST egg lay was decreased (P<0.0088) by nearly six days in inhibin-treated hens (Table 1). Likewise, the age to FIFTY egg production was markedly reduced (12 days; P<0.01) in inhibin-treated hens (Table 2).

A positive effect of inhibin treatment on hen day egg production (HDEP) was also extant, most notably at the beginning and at the end of the laying cycle. For example, significantly greater (P<0.05) mean HDEP rates were observed in hens treated with MBP-cINA₅₂₁/FRN when compared to the FRN controls during Weeks 1 (16.5 vs 2.6%), 2 (50.0 vs 28.6%), and 4 (96.6 vs 79.7%) and again during Weeks 15 (98.8 vs 86.9%), 16 (96.9 vs 86.3%), 18 (85.7 vs 66.1%), and 20 (96.8% vs 73.8%). Total HDEP rate (inclusive of all 20 weeks of lay) for inhibin-treated hens was 83.5% as compared to 75.4% for the controls.

Besides accelerating puberty, prolonging egg lay, and enhancing the overall intensity of lay, inhibin-treatment decreased the time needed to reach peak egg lay by approximately 3 weeks. MBP-cINA₅₂₁/FRN had HDEP of 96.6% by Week 4 while FRN had HDEP of 96.6% by Week 7). Although differences in peak HDEP values were not statistically evaluated, the treatment differences in mean age at which hens reached 50% HDEP levels (FIFTY) reflect peak performance.

Mortality was not a factor in this study as only eight birds have died (three controls, five treated). Such MORT (16%) would be within expected limits for quail that have reached 180 days-of-age.

TABLE 1
Effect of inhibin immunoneutralization on mean
(±SE) age at first egg lay in Japanese quail

| Treatment | Age at first egg lay (days) |
|---|---|
| FRN¹ | 56.15 ± 1.82^{a} |
| MBP-cINA₅₂₁/FRN² | 50.38 ± 1.08^{b} |

¹= Freund's adjuvant control.
²= MBP-cINA₅₂₁/FRN = Maltose Binding Protein-chicken α515-inhibin fusion protein in Freund's adjuvant.
^{a,b}(p < .0088).

TABLE 2
Effect of inhibin immunoneutralization on mean
(±SE) age at 50% egg production in Japanese quail

| | Age at 50% |
|---|---|
| | egg production |
| Treatment | (days) |
| FRN¹ | 73.04 ± 3.78^{a} |
| MBP-cINA₅₂₁/FRN² | 61.00 ± 2.70^{b} |

¹= Freund's adjuvant control.
²= MBP-cINA₅₂₁/FRN = Maltose Binding Protein-chicken α515-inhibin fusion protein in Freund's adjuvant.
^{a,b}(p < .01).

Incidences of shelless (unshelled) and thin-shelled eggs occurred at greater frequencies in control birds, particularly during the latter stages of the laying cycle, than in inhibin-immunized birds. This suggests that greater numbers of defective eggs (i.e., eggs that were either unfit for consumption or likely to break before consumption, or unsettable as hatching eggs) were associated with the control treatment.

### EXAMPLE 4

Enhancing Fertility and/or Growth Rate in Chickens.

The protein conjugate (MBP-cINA₅₂₁) is used as an antigen to immunize prepubescent, female chickens against circulating inhibin levels, and to therefore accelerate the onset of egg lay in the treated chickens. The method described in Example 8 is followed with the following exceptions. The average age at puberty for an untreated chicken is approximately 20 weeks. The following is a treatment schedule for a chicken having an approximate body weight range of 2.0 to 3.5 pounds: primary (first) injection of 1.5 mg of the heterologous protein of the present invention on its 15th week of age; and a booster of 0.75 mg on the 17th week. Another treatment schedule for egg-type chickens having the same body weight is: primary (first) injection of 1.5 mg of the heterologous protein of the present invention on its 15th week of age; and boosters of 0.375 mg on the 17th, 20th, 24th, 30th, 40th, and 50th week of age. For meat-type chickens, the average age at puberty for an untreated chicken is approximately 23-25 weeks. The following would be a treatment schedule for a meat-type chicken having an approximate body weight range of 3.25 to 4.0 pounds at primary injection: primary (first) injection of 1.5 mg of the heterologous protein of the present invention in its 18th week of age; and a booster of 0.75 mg at the 20th week. Another treatment schedule for egg-type chickens having the same body weight is: primary (first) injection of 1.5 mg of the heterologous protein of the present invention on its 18th week of age; and boosters of 0.75 mg on the 20th, 24th, 30th, 40th, and 50th week of age.

### EXAMPLE 5

Enhancing Fertility and/or Growth Rate in Turkeys.

The protein conjugate (MBP-cINA₅₂₁) is used as an antigen to immunize prepubescent, female turkeys against circulating inhibin levels, and to therefore accelerate the onset of egg lay in the treated turkeys. The method described in Example 8 is followed with the following exceptions. The average age at puberty for an untreated turkey is approximately 30 weeks. The following is a treatment schedule for a turkey having an approximate body weight range of 9.0 to 12 pounds: primary (first) injection of 2.0 mg of the heterologous protein of the present invention on its 28th week of age; and a booster of 1.0 mg on the 29th week of age. Another treatment schedule for a turkey having the same weight is: primary (first) injection of 2.0 mg of the heterologous protein of the present invention on its 28th week of age; and boosters of 1.0 mg on the 29th, 30th, 34th, 38th, 46th, and 54th week of age.

### EXAMPLE 6

Enhancing Body Weight, Testes Weight, and Plasma Testosterone in Male Broiler Breeder Chickens

The effect of inhibin immunoneutralization on the fertility and/or growth rate of male broiler breeders was examined during the selected time periods of early puberty (24 weeks of age), peak sexual productivity (28 weeks of age), and sexual decline (39 weeks of age). Male broiler breeders (n=288, Cobb 500) were obtained from a commercial source (Tyson Foods, Jacksonville, Fla.). Up to 6 weeks of age, birds were fed a breeder starter ration (18-21.0% CP; 2915 kcal ME/kg). At week 7, birds were placed on a grower ration (15.0% CP; 2860 kcal ME/kg). Industry standard vaccination treatments were administered between 4-10 weeks of age. At 12 weeks, 2 days of age, birds were transferred to a light-tight, environmentally controlled house at the Louisiana State University (LSU) Poultry Farm, (Baton Rouge, La.). Birds were randomly distributed into 18 pens (16 birds/pen). Each pen measured 1.5×3.0 m (L×W). Wood shavings served as a floor substrate. A diet similar in protein and ME content to that used before transfer was continued at LSU until 18 weeks of age. At 18 weeks, birds were given a breeder ration (16.0% CP; 2870 kcal ME/kg) in accordance with the Cobb-500 Breeder Management Guide (ED limited feeding). They remained on this ration until the end of the study (39 weeks of age). There were two tube-type feeders per pen. Birds were supplied ad libitum with water via a drip-nipple watering-system, 6 nipples per pen. Birds were kept on an 8-h photoperiod until 20 weeks of age. At that time, light was increased to 13 hours/day. Light was further increased, thereafter, at a rate of 1 hour per week until a maximum of 16 hours was achieved. At 13 weeks, birds were randomly assigned to one of four treatment groups (n=72 birds/treatment) such that each pen included four birds from each treatment group. The groups were defined based upon receipt of a subcutaneous, primary inoculation of 0, 1.0, 3.0, or 5.0 milligrams per bird of the inhibin-based immunogen, MBP-cINA₅₂₁, emulsified in Freund's Complete Adjuvant (see below). (The groups will be referred to herein as the CON, 1-mg, 3-mg, and 5-mg groups, respectively). CON received Freund's Complete Adjuvant without MBP-cINA₅₂₁. Prior to inoculation, birds within a pen were randomly selected, weighed (BWT), and fitted with color-coded wing bands to identify them by treatment group. After inoculation, birds were returned to their respective home pens, such that all injection treatments were represented by an equal number of birds (4) within each of the 18 pens. Booster vaccinations with the MBP-cINA₅₂₁ immunogen, given at 18 weeks of age, consisted of one-half the primary dosage emulsified in Freund's Incomplete Adjuvant. CON received a booster injection of Freund's Incomplete Adjuvant with no MBP-cINA_{521.}

At 24 weeks of age, 24 birds of each treatment group were randomly selected, weighed (BWT), blood sampled, and sacrificed. With respect to mortality, there was a progressive decline in livability with advancing age due to an expected, age-related heightened aggression (fighting) associated with housing breeder males without females in close quarters. Thus, at 28 and 39 weeks of age, essentially equal but progressively lower numbers of remaining birds per treatment were weighed (BWT), blood sampled, and sacrificed. The treatment sample sizes at the 28 weeks and 39 weeks of age time points varied as, in effect, all mortalities were absorbed in these time points. The numbers of males tested by treatment at 28 weeks (out of the original number of 24 birds per treatment that would have been used for the 28 week point) were 17, 17, 15 and 18 birds for the 0, 1, 3 and 5-mg MBP-cINA₅₂₁ treatments, respectively. The numbers of males alive by treatment at 39 weeks (out of the original number of 24 birds per treatment would have been used for the 39 week point) were 11, 11, 12 and 14 birds for the 0, 1, 3 and 5-mg MBP-cINA₅₂₁ treatments, respectively. Despite the mortality evident with aging, there were no differences in average MORT rates between treatment groups at any of the study intervals (24, 28 and 39 weeks of age). For each bird sacrificed at each interval, testes were removed by blunt dissection and the wet weights (nearest 0.01 g) of the left and right testis were combined to give a measure of total testes weight (TWT). In addition, for each bird sacrificed at each interval, the blood sample was used to determine plasma testosterone (T) levels using a radioimmunoassay (Coat-A-Count kit, Diagnostic Products Company, Los Angeles, Calif.). Daily mortality (MORT) records were kept throughout the study.

The influence of injection treatments on BWT, TWT, and plasma T at each study interval (24, 28 and 39 weeks of age) were assessed by independent one-way ANOVAs each using a completely randomized design (CRD). An additional CRD ANOVA to detect BWT differences in the birds that were randomly selected for assignment into the injection treatment groups was conducted at 13 weeks of age (i.e., BWT prior to primary inoculations). Post-hoc differences in injection treatment means were detected by use of Duncan's New Multiple Range Test.

Random assignment of males into treatment groups at 13 weeks of age (prior to primary inoculations) resulted in no differences in mean BWT at 13 weeks. The CON, 1.0, 3.0 and 5.0 mg MBP-cINA₅₂₁ groups had average BWT of 2098, 2078, 2032 and 2047 grams, respectively at 13 weeks.

Body weight (BWT), results are presented in Table 3.

TABLE 3
Male Inhibin Trial:

| | 24 wk of age | | | | | | | | 39 wk of age | | | | |
|---|---|---|---|---|---|---|---|---|---|---|---|---|---|
| | | | | Statistical | P | 28 wk of age | | | | | | Statistical | P |
| Dose | N | Mean | SE | Differences | Level | N | Mean | SE | N | Mean | SE | Differences | Level |

Body Weight (g)

| | | | | | | | | | | | | | |
|---|---|---|---|---|---|---|---|---|---|---|---|---|---|
| 0 | 24 | 2664 | 88 | a | <0.05 | 17 | 3771 | 99 | 11 | 4152 | 173 | a | <0.10 |
| 1 | 21 | 2862 | 129 | a, b | | 16 | 3649 | 135 | 11 | 4655 | 248 | b | |
| 3 | 24 | 2887 | 106 | a, b | | 14 | 3661 | 110 | 12 | 4249 | 215 | a, b | |
| 5 | 23 | 3041 | 123 | b | | 18 | 3735 | 109 | 14 | 4144 | 131 | a | |

Testes Weight (g)

| | | | | | | | | | | | | | |
|---|---|---|---|---|---|---|---|---|---|---|---|---|---|
| 0 | 24 | 8.3 | 1.5 | a | <0.05 | 17 | 35.9 | 3.0 | 11 | 20.0 | 3.2 | a | <0.10 |
| 1 | 23 | 12.4 | 2.1 | a, b | | 17 | 34.3 | 2.2 | 11 | 28.5 | 3.6 | b | |
| 3 | 24 | 12.0 | 1.9 | a, b | | 15 | 36.7 | 2.7 | 12 | 26.0 | 2.9 | a, b | |
| 5 | 23 | 14.0 | 1.7 | b | | 18 | 34.1 | 2.3 | 14 | 27.5 | 2.7 | a, b | |

Plasma Testosterone (ng/mL)

| | | | | | | | | | | | | | |
|---|---|---|---|---|---|---|---|---|---|---|---|---|---|
| 0 | 24 | 0.16 | 0.06 | a | <0.05 | 16 | 1.13 | 0.38 | 11 | 0.19 | 0.11 | a | <0.10 |
| 1 | 22 | 0.22 | 0.13 | a | | 17 | 1.41 | 0.40 | 11 | 0.79 | 0.35 | b | |
| 3 | 24 | 0.40 | 0.10 | a, b | | 15 | 0.60 | 0.30 | 12 | 0.37 | 0.13 | a, b | |
| 5 | 23 | 0.59 | 0.17 | b | | 18 | 1.11 | 0.34 | 14 | 0.42 | 0.17 | a, b | |

At 24 weeks of age, the mean BWT for birds given 5-mg of MBP-cINA₅₂₁ was significantly higher (P<0.05) than the mean BWT of the CON, and the 1-mg and 3-mg dose groups had average BWT values that were intermediate between the control and 5-mg dose groups. In aged males (39 weeks of age), birds that received 1-mg of the immunogen had a significantly higher (P<0.10) mean BWT than either the CON or birds given the 5-mg dose. The mean BWT of males given 3-mg of MBP-cINA₅₂₁ was intermediate between BWT found in the CON and those birds given the 1 mg dose.

Total Testes Weight (TWT) results are presented in Table 3. At 24 weeks of age, mean TWT at this time was significantly higher (P<0.05) for males given the 5-mg dose of MBP-cINA₅₂₁ when compared to CON. The 1-mg and 3-mg dosage groups had mean TWT that were intermediate between those of the CON or the 5-mg group. At 39 weeks, when reproductive senility would be expected, birds that received 1 mg of the immunogen had a significantly greater (P<0.10) mean TWT (43% enhancement) when compared to the CON. Birds treated with the two highest dosages of MBP-cINA₅₂₁ also had TWTs that were markedly higher (3-mg, 30%; 5-mg, 38%) than the CON.

Further analysis of TWT, specifically, the percentage of males in each group having a TWT over 20 grams is shown in Table 4. 20 grams was selected because 10 grams per individual testis is the threshold weight for normal sperm production for a fertile male. Vizcarra, J. A. et al., “Physical Factors Affecting the Reproductive Performance of Commercial Broiler Breeder Males,” Proceedings from the 49^{th} Annual National Breeders Roundtable Association, May 4-5, 2000 (Poultry Breeders of America and U.S. Poultry and Egg Association).

TABLE 4
% males with Total Testes Weight over 20 grams

| | 24 | 28 | 39 |
|---|---|---|---|
| | Weeks | Weeks | Weeks |
| Control | 8.3 | 94.1 | 36.4 |
| 1 mg MBP-cINA₅₂₁ | 33.3 | 94.1 | 81.8 |
| 3 mg MBP-cINA₅₂₁ | 20.8 | 93.3 | 75 |
| 5 mg MBP-cINA₅₂₁ | 30.4 | 88.9 | 85.7 |

At 24 weeks of age, the percentage of males with TWT over 20 grams was significantly higher for males given the 1-mg dose group (P=0.06) and 5-mg dose group (P<0.05) when compared to CON. The 3-mg dose group was intermediate between the 1-mg does group and the CON. At 39 weeks of age, the percentage of males with TWT over 20 grams was significantly higher for males given the 1-mg dose group and the 5-mg dose group (both P<0.05) as well as the 3-mg dose group (P=0.07) when compared to CON. Because spermatozoa production is directly related to testicular size in avian males (de Reviers et al., 1988), the increase in TWT in immunized birds indicates an enhanced spermatozoa production rate.

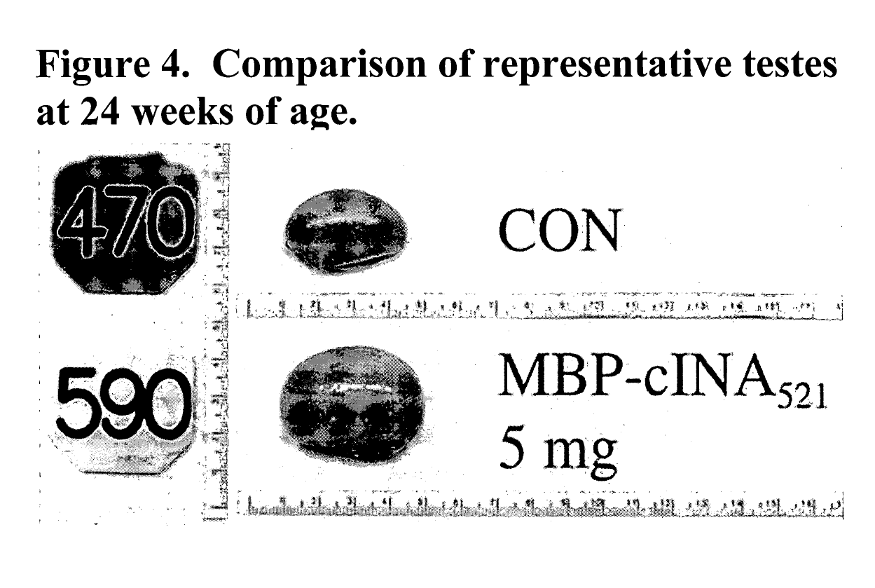

Plasma Testosterone (T) levels are presented in Table 3. At 24 weeks, mean plasma T levels were significantly elevated (P<0.05) in the 5-mg dose group when compared to the mean plasma T levels in the CON and 1-mg dose groups (FIG. 4). Treatment with 3-mg of the immunogen yielded an intermediate plasma T response. At 39 weeks of age, although plasma T levels had dramatically declined in the CON, higher mean plasma T levels remained extant in immunized birds. Birds of the 1-mg dose group had mean plasma T levels that were more than four-fold greater (P<0.10) than those of the CON; and, the 3- and 5-mg treatments showed responses that were intermediate between those of the CON and the 1-mg groups but were both about 2-fold higher than those of the CON.

The results at 24 weeks indicate that vaccination with the MBP-cINA₅₂₁ immunogen resulted in an acceleration in somatic growth rate as well as testes growth rate and testosterone production in male birds. This suggests an acceleration in puberty. By 39 weeks of age, as expected, CON males TWT had decreased to 20.0 g. In contrast, all three immunized bird groups enjoyed average TWT of nearly 30 g. Moreover, plasma T at 39 wk in the 1-, 3- and 5-mg groups were, respectively, 410, 180, and 218% greater than those found in the CON. These TWT and plasma T findings in aged breeders suggest that MBP-cINA₅₂₁ immunoneutralization significantly slowed sexual senescence of males. Because most of the overall mortalities were absorbed at the 39 weeks of age time point, the reduced number of observations within treatment groups at this time point likely precluded the finding of stronger P-values in the analyses of BWT, TWT and plasma T differences between treatment groups in this study. Nevertheless, certain immunization-induced enhancements in all three variables (with a 90% degree of certainty, P<0.10) were again evident at 39 wk of age, particularly for TWT and plasma T levels.

It should be understood, of course, that the foregoing relates only to preferred embodiments of the present invention and that numerous modifications or alterations may be made therein without departing from the spirit and the scope of the invention as set forth in the appended claims.

What is claimed is:

1. A method of increasing growth rate or enhancing fertility of a bird, comprising administration of an effective amount of a composition comprising a heterologous protein comprising inhibin protein, or a fragment thereof, and a carrier protein, to the bird.

2. The method of claim 1, wherein the bird is male and enhancing fertility is accelerating onset of puberty, accelerating onset of sperm production, increasing intensity of sperm production, accelerating onset of maximum sperm production, increasing daily sperm production, accelerating onset of maximum daily sperm production, prolonging persistence of sperm production, improving sperm viability, increasing testosterone production, increasing ejaculate volume, increasing libido, increasing testes growth rate, increasing testes weight, accelerating an increase of plasma testosterone levels during growth and puberty, increasing the lifetime sperm production, delaying reproductive senescence, delaying decline of testes weight in older males, delaying decline of plasma testosterone levels in older males, delaying the decline of sperm production in older males, increasing sperm motility, increased sperm mobility, improving copulation efficiency, improving lifetime fertilization capacity, or a combination thereof.

3. The method of claim 1, wherein the inhibin protein, or the fragment thereof, has or contains a substantially similar sequence to SEQ ID NO: 2.

4. The method claim 1, wherein the bird is selected from the group consisting of ratites, psittaciformes, falconiformes, piciformes, strigiformes, passeriformes, coraciformes, ralliformes, cuculiformes, columbiformes, galliformes, anseriformes, and herodiones.

5. The method of claim 1, wherein the bird is a poultry bird.

6. The method claim 1, wherein the bird is a chicken, turkey, parrot, parakeet, makaw, falcon, eagle, quail, hawk, pigeon, cockatoo, song bird, jay bird, blackbird, finch, warbler, goose, duck, canary, mynah, toucan, or sparrow.

7. The method of claim 1, wherein the bird is a chicken, turkey, quail, goose or duck.

8. The method of claim 1 wherein the administration of an effective amount of the protein comprises an initial immunization followed by one or more booster injections.

9. The method of claim 1, wherein the carrier protein is maltose binding protein, bovine serum albumin, ovalbumin, flagellin, keyhole limpet hemocyanin, thyroglobulin, serum albumin, gamma globulin, or polymers of amino acids.

10. The method of claim 1, wherein the effective amount is approximately 0.1 mg to approximately 10.0 mg, approximately 1 mg to approximately 5 mg, approximately 0.3 mg to approximately 5.0 mg, approximately 0.3 mg to approximately 3.0 mg, approximately 0.3 mg to approximately 2.0 mg or approximately 0.3 mg to approximately 1.5 mg.

11. The method of claim 1, wherein the heterologous protein is a fused heterologous protein.

12. The method of claim 1, wherein the heterologous protein is a conjugated heterologous protein.

13. The method of claim 1, further comprising administration of adjuvants, preservatives, diluents, emulsifiers, or stabilizers.

14. The method of claim 1, wherein the inhibin is bird inhibin, fish inhibin, reptile inhibin, amphibian inhibin, mammal inhibin, cow inhibin, human inhibin, horse inhibin, cat inhibin, dog inhibin, rabbit inhibin, sheep inhibin, mink inhibin, fox inhibin, otter inhibin, ferret inhibin, raccoon inhibin, donkey inhibin, rat inhibin, mouse inhibin, hamster inhibin, or pig inhibin.

15. The method of claim 1, wherein the inhibin protein or the fragment thereof is alpha-subunit inhibin or a fragment thereof.

16. The method of claim 1, wherein increasing growth rate comprises increasing body weight, increasing skeletal growth, increasing the rate at which the animal adds muscle mass, or a combination thereof.


SEQUENCE LISTING (4 sequences)

SEQ ID NO: 1 - DNA, 521 bp, Gallus sp.
  CDS (1)..(303)
ctgcagcgcccatcggaggacgtggccgcccacaccaactgccgccgggcgtccctcaacatctctttcgaggagctgggctgggacaattggatcgtgcaccccagcagcttcgttttccactactgccacgggaactgtgccgaaggccacgggctgagccaccggctgggggtgcagctgtgctgcgccgcgctgcccggcaccatgcgctcactgcgtgtccgcaccacctctgatggtggctactccttcaagtacgagacggtgcccaacatcctggcgcaggactgcacctgtgtctagcagctggcatgcacggccagacccgcgtggatctccccgttgcctctggactgccccagtgccagatgatgagcccatcccagggatggaggagtcactcacacgggcactgcgcagcccggagcagggagagggacccaggtggaagttttggtggtgccaccctccctttgactgccagggtttcatggtttcaggttgcgtgggtgctgcag

SEQ ID NO: 2 - protein, 101 aa, Gallus sp.
LQRPSEDVAAHTNCRRASLNISFEELGWDNWIVHPSSFVFHYCHGNCAEGHGLSHRLGVQLCCAALPGTMRSLRVRTTSDGGYSFKYETVPNILAQDCTCV

SEQ ID NO: 3 - DNA, 20 bp, Artificial Sequence
  Synthetic
ctcagcctgctgcagcgccc

SEQ ID NO: 4 - DNA, 22 bp, Artificial Sequence
  Synthetic
gtgtcgaccgcgcgacgccgac